(19) United States
(12) Patent Application Publication
(10) Pub. No.: US 2011/0257145 A1
(43) Pub. Date: Oct. 20, 2011

(54) METHOD OF TREATING NEUROLOGICAL DISEASES

(75) Inventors: Alan Kozikowski, Chicago, IL (US); Yihua Chen, Shanghai (CN)

(73) Assignee: THE BOARD OF TRUSTEES OF THE UNIVERSITY OF ILLINOIS, Urbana, IL (US)

(21) Appl. No.: 13/056,821

(22) PCT Filed: Aug. 5, 2009
(86) PCT No.: PCT/US09/52809

§ 371 Date: Jul. 8, 2011

Related U.S. Application Data
(60) Provisional application No. 61/086,220, filed on Aug. 5, 2008.

(51) Int. Cl. A61K 31/395; A61P 25/28; A61P 25/00; A61P 25/14; A61P 25/24; A61P 9/10; A61P 21/00; A61P 25/16; A61P 25/22; C07D 245/06; A61P 25/18

(52) U.S. Cl. 514/183; 540/460

(57) ABSTRACT

A method of treating neurological diseases, like Alzheimer's Disease by administering a compound that activates protein kinase C and inhibits histone deacetylases to an individual suffering from the neurological disease. The method utilizes benzolactam compounds that reduce Aβ production and blocks oxidative stress in the treatment of Alzheimer's disease. The benzolactam compounds and compositions containing the benzolactam compounds also are disclosed.

### STATEMENT OF GOVERNMENTAL INTEREST

This application claims the benefit of U.S. provisional application No. 61/086,220, filed Aug. 5, 2008, incorporated herein by reference in its entirety.

### STATEMENT OF GOVERNMENTAL INTEREST

This invention was made with government support under Grant No. R01AG022941 awarded by the National Institutes of Health. The government has certain rights in the invention.

### FIELD OF THE INVENTION

The present method is directed to a method of treating neurological diseases comprising administering a therapeutically effective amount of a compound that both activates protein kinase C (PKC) and inhibits histone deacetylase (HDAC) to an individual in need thereof. More particularly, the present invention is directed to a method of treating Alzheimer's Disease (AD) comprising administering a therapeutically effective amount of a benzolactam compound to an individual in need thereof. The present invention also relates to the benzolactam compounds and to compositions comprising the benzolactam compounds.

### BACKGROUND OF THE INVENTION

Alzheimer's disease (AD) is a devastating neurological disorder characterized by deteriorating cognition and memory, progressive impairment in the ability to carry out daily living activities, and a number of neuropsychiatric symptoms. Associated with this progressive deterioration is the pathological generation and accumulation of Aβ₄₀₋₄₂ plaques, which gives rise to the amyloid hypothesis, i.e., that the pathological accumulation of Aβ₄₀₋₄₂ in the brain leads to oxidative stress, neuronal destruction, and finally the clinical syndrome of AD.

As a result of this hypothesis, strategies to decrease the production of Aβ₄₀₋₄₂, to stimulate the clearance of formed Aβ, and/or to prevent the aggregation of Aβ into amyloid plaques have been investigated. However, these strategies failed to recognize and treat other factors that are concomitant with AD pathology, such as oxidative stress, neuronal degeneration, and impaired memory and learning.

Therefore, a strategy that targets multiple pathological processes in AD should be considered. One strategy is combination therapy. Combination therapy using two or more drugs has been used successfully in the treatment of cancer, HIV infection, and tuberculosis. An alternative to combination therapy is to identify a single compound that acts on and/or interdicts in multiple relevant processes.

In the identification of a single compound that acts on multiple processes relevant to AD, there is an interest in protein kinase C (PKC), which plays a critical role in memory [1] and amyloid precursor protein (APP) processing [2-4]. PKC activity also has been found to be altered or defective in brains [5-10] and peripheral tissues of AD patients [11,12]. PKC also plays a role in APP processing because of its ability to modulate the activity of α-secretase, a metalloprotease that has been neither completely identified nor characterized [2-4]. Activation of a-secretase shifts formation of the amyloidogenic Aβ₄₀₋₄₂, generated by β- and γ-secretases, to generation of the non-amyloidogenic sAPPα. Because Aβ plaque formation in the brain is important in the etiology of Alzheimer's pathophysiology [13,14], suppression of neurotoxic Aβ₄₀ and/or Aβ₄₂ formation by PKC activation represents a non-protease-based approach to controlling Aβ levels.

It previously was demonstrated that 8-(1-decynyl)benzolactam (BL), a PKC activator, reverses K⁺ channel defects and enhances the secretion of sAPPα in AD cells [15, 16]. Another PKC activator, bryostatin 1, at sub-nanomolar concentrations, dramatically enhances the secretion of the a-secretase product sAPPα in fibroblasts from AD patients. Moreover, BL was found to significantly increase the amount of sAPPα and decrease Aβ₄₀ in the brains of APP-[V717I] transgenic mice in vivo [17]. In an AD double-transgenic mouse, bryostatin 1 decreased both brain Aβ₄₀ and Aβ₄₂, ameliorated the rate of premature death, and improved behavioral outcome [17]. These findings indicate that PKC and PKC activation potentially is a target for ameliorating AD pathophysiology and cognitive impairment.

U.S. patent publication No. 2003/0050302 discloses benzolactam compounds useful in the treatment of Alzheimer's Disease. WO 97/43268 discloses benzolactam compounds that modulate PKC activity.

Another approach to affect multiple processes to ameliorate AD pathophysiology and cognitive impairment using a single molecule is epigenetic remodeling through the inhibition of histone deacetylase (HDAC). Acetylation and deacetylation of histone proteins play critical roles in transcriptional regulation in eukaryotic cells. The acetylation status is determined by the activities of two families of enzymes, histone acetyltransferases (HATs), which add acetyl groups to conserved lysine residues within the amino-terminal tails of histone proteins, and HDACs, which remove these acetyl groups.

In general, acetylation of histone promotes a more relaxed chromatin structure, allowing transcriptional activation. HDACs are able to act as transcriptional repressors because histone deacetylation consequently promotes chromatin condensation. HDAC inhibitors selectively alter gene transcription, in part, by permitting chromatin remodeling by HAT activity and changes to the composition of multiprotein complexes bound to the proximal region of specific gene promoters [18]. Furthermore, HDACs interact with many non-histone protein substrates, such as hormone receptors, chaperone proteins, and cytoskeletal proteins, which regulate cell proliferation and cell death [19, 20].

HDAC inhibitors are neuroprotective in a broad array of cellular and animal models of acute and chronic neurodegenerative injury and disease, including AD, Huntington's disease (HD), spinal muscular atrophy (SMA) and spinal and bulbar muscular atrophy (SBMA), amyotrophic lateral sclerosis (ALS), ischemic stroke, and experimental autoimmune encephalomyelitis (EAE) [21, 22]. At least part of this broad neuroprotective capacity centers on the ability of HDAC inhibitors to increase neuronal resistance to oxidative stress, a pathological process that underlies many neurodegenerative disease pathologies, and that is particularly relevant to AD.

In addition to neuroprotection, HDAC inhibition enhances synaptic plasticity, learning, and memory in rodents. Furthermore, in a transgenic mouse model (CK-p25Tg) that allows the temporal and spatial induction of neuronal degeneration and displays many of the pathologic hallmarks of AD, HDAC inhibition significantly improved associative and spatial learning after degeneration [23]. Importantly, HDAC inhibition can facilitate the recovery of inaccessible long-term memories in this model [23].

Based on research directed to PKC activators and HDAC inhibitors in AD and other neurodegenerative diseases, it would be an advance in the art to utilize a single chemical compound capable of targeting both PKC and HDAC enzymes simultaneously, and administer this compound in a method of treating AD and related neurological diseases.

### SUMMARY OF THE INVENTION

The present invention relates to benzolactam compounds capable of activating PKC and inhibiting HDAC, and to methods of treating neurological diseases comprising administering a therapeutically effective amount of the benzolactam compound to an individual in need thereof. The present invention also relates to compositions and kits containing the benzolactam compounds.

In particular, the present invention is directed to benzolactam compounds and to a method of treating neurological diseases, such as Alzheimer's Disease, comprising administering a therapeutically effective amount of a benzolactam of structural formula (I) to an individual in need thereof,

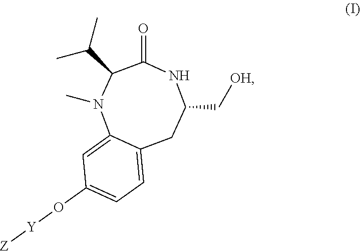

wherein Y is selected from the group consisting of null, C₁₋₈alkylene, NR^{b}, C(═O), C(═O)C₁₋₆alkylene, C₁₋₈alkyleneNR^{b}, C₁₋₄alkylenearyleneC₁₋₄alkylene, C₂₋₆alkenylene, C₄₋₈alkdienylene, C₁₋₆alkylenearylene, C(═O) arylene, C(═O)C₁₋₆alkylenearylene, C₁₋₆alkylenepiperidinyl, and C₂₋₆alkenylenearyleneC₁₋₄alkylene; and
Z is selected from the group consisting of

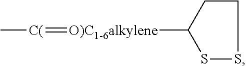

—C₃₋₁₀alkyleneSAc, —C(═O)N(R^{c})OH,

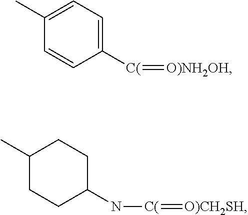

—SO₂NHR^{c}, —NHSO₂NHR^{c}, —NHSO₂C₁₋₆alkyl, —SO₂C₁₋₆alkyl,

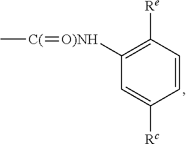

—C(═O)R^{f} wherein R^{f} is selected from the group consisting of OH, N(R^{c})², NH(OCH₃), N(CH₃)OH, C₁₋₆alkyl, CF₃, aryl, heteroaryl, C₃₋₈cycloalkyl, heterocycloalkyl, NHSO₂CH₃, NHSO₂CF₃, and C₁₋₆haloalkyl, —C(═O)(C(R^{c})₂)₁₋₃SH, —SR^{d} wherein R^{d} is hydrogen or (C═O)CH₃,

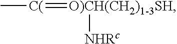

—S—(C═O)C₁₋₆alkyl, heterocycloalkyl optionally substituted with oxo (═O), thioxo (═S), or both, heteroaryl optionally substituted with —NH₂, —SH, or both, —N(H)C(═O)SH, —NHC(═O)NHR^{e}, —NHC(═O)CH₂R^{e}, —NHC(═O)(CH₂)₁₋₆SH, —NHC(═O)CH₂Hal, —NHC(═S)NHR^{e}, —NHC(═S)CH₂R^{e}, —C(═S)NHR^{e}, —C(═S)CH₂R^{e}, —NHC(═S)CH₂R^{e}, —NHC(═S)CH₂Hal, and —(C═O)C₁₋₃alkyl;
R^{b}, independently, is selected from the group consisting of hydrogen, C₁₋₆alkyl, aryl, heteroaryl, C₃₋₈cycloalkyl, and heterocycloalkyl;
R^{c}, independently, is selected from the group consisting of hydrogen, (C═O)CH₃, C₁₋₆alkyl, CF₃, CH₂F, and aryl, or two R^{c} groups are taken together with the carbon to which they attached to form a C₃₋₈cycloalkyl group; and
R^{e} is NH₂ or OH;
or a pharmaceutically acceptable salt, hydrate, or prodrug thereof.

In another embodiment, the present invention provides a method of treating a neurological disease comprising administering to an individual in need thereof, such as a human, a therapeutically effective amount of a benzolactam compound of structural formula (I). In all embodiments, a benzolactam can be the sole therapeutic agent or can be administered with additional therapeutic agents known to treat the disease or condition of interest.

In another embodiment, the present invention provides a method of altering conditions associated with amyloid processing in order to enhance an α-secretase pathway to generate soluble α-amyloid precursor protein (α-APP), and thereby prevent β-amyloid aggregation, comprising administering a biologically effective amount of a benzolactam of structural formula (I).

In another embodiment, the present invention also provides a pharmaceutical composition comprising a benzolactam of structural formula (I) and a pharmaceutically acceptable excipient.

Another embodiment of the present invention is to utilize a benzolactam of structural formula (I) and an optional second therapeutically active agent useful in the treatment of Alzheimer's Disease in a method of treating an individual for Alzheimer's Disease.

In a further embodiment, the invention provides for use of a composition comprising an benzolactam of structural formula (I) and an optional second therapeutic agent for the manufacture of a medicament for treating a disease or condition of interest, e.g., Alzheimer's Disease.

Still another embodiment of the present invention is to provide a kit for human pharmaceutical use comprising (a) a container, (b1) a packaged composition comprising a benzolactam of structural formula (I), and, optionally, (b2) a packaged composition comprising a second therapeutic agent useful in the treatment of a disease or condition of interest, and (c) a package insert containing directions for use of the composition or compositions, administered simultaneously or sequentially, in the treatment of the disease or condition of interest.

The benzolactam of structural formula (I) and the second therapeutic agent can be administered together as a single-unit dose or separately as multi-unit doses, wherein the benzolactam of structural formula (I) is administered before the second therapeutic agent, or vice versa. It is envisioned that one or more dose of a benzolactam of structural formula (I) and/or one or more dose of a second therapeutic agent can be administered.

In one embodiment, a benzolactam of structural formula (I) and a second therapeutic agent are administered simultaneously. In related embodiments, a benzolactam of structural formula (I) and second therapeutic agent are administered from a single composition or from separate compositions. In a further embodiment, a benzolactam of structural formula (I) and a second therapeutic agent are administered sequentially. A benzolactam of structural formula (I) can be administered in an amount of about 0.005 to about 500 milligrams per dose, about 0.05 to about 250 milligrams per dose, or about 0.5 to about 100 milligrams per dose.

The benzolactam compounds of the invention activate PKC and inhibit HDAC, and are useful research tools for in vitro study of protein kinase C and histone deacetylases and their role in biological processes.

These and other novel aspects of the present invention will become apparent from the following detailed description of the preferred embodiments.

### BRIEF DESCRIPTION OF THE DRAWINGS

FIG. 1 contains bar graphs showing the effect of compounds 9-14 (in c/μM) on SAPPα levels;

FIG. 2 contains bar graphs showing the effect of compounds 9-14 (in c/μM) on decreasing Aβ production;

FIG. 3 contains Western blots of a control, TSA, SAHA, and compounds 3, 6, 7, and 9-14 (in μM) showing the effect on acetylation levels of histone H4; and

FIG. 4 contains bar graphs showing the dose dependent survival of cortical neurons upon exposure to compounds 2, 3, 6, 7, 9-14, TSA, and SAHA.

### DETAILED DESCRIPTION OF THE PREFERRED EMBODIMENTS

The present invention is directed to novel benzolactams and their use in therapeutic treatments of neurological diseases, such as Alzheimer's Disease. The present benzolactams activate PKC and inhibit HDAC.

The present invention is described in connection with preferred embodiments. However, it should be appreciated that the invention is not limited to the disclosed embodiments. It is understood that, given the description of the embodiments of the invention herein, various modifications can be made by a person skilled in the art. Such modifications are encompassed by the claims below.

The term “a disease or condition wherein activation of PKC and inhibition of HDAC provides a benefit” pertains to a condition in which PKC activation and HDAC inhibition is important or necessary, e.g., for the onset, progress, and/or expression of that disease or condition. Examples of such conditions include, but are not limited to, neurological diseases, e.g., Alzheimer's Disease, Parkinson's Disease, Huntington's chorea, amyotropic lateral sclerosis, and spino-cerebellar degeneration. One of ordinary skill in the art is readily able to determine whether a compound treats a disease or condition mediated by PKC and HDAC for any particular cell type, for example, by assays which conveniently can be used to assess the activity of particular compounds.

The term “PKC” refers to a family of enzymes involved in controlling the function of other proteins through phosphorylation of the hydroxyl groups of serine and threonine amino acid residues on these proteins. PKC enzymes are activated by signals, such as increases in the concentration of diacylglycerol (DAG). The PKC family of enzymes consists of at least eleven isozymes. As used herein, PKC refers to the entire family of isoforms.

The term “HDAC” refers to a family of enzymes that remove acetyl groups from a protein, for example, the c-amino groups of lysine residues at the N-terminus of a histone. The HDAC can be a human HDAC, including HDAC1, HDAC2, HDAC3, HDAC4, HDAC5, HDAC6, HDAC7, HDAC8, HDAC9, HDAC10, and HDAC11. The HDAC also can be derived from a protozoal or fungal source.

As used herein, the terms “treat,” “treating,” “treatment,” and the like refer to eliminating, reducing, relieving, reversing, or ameliorating a disease or condition and/or symptoms associated therewith. Although not precluded, treating a disease or condition does not require that the disease, condition, or symptoms associated therewith be completely eliminated. As used herein, the terms “treat,” “treating,” “treatment,” and the like may include “prophylactic treatment,” which refers to reducing the probability of redeveloping a disease or condition, or of a recurrence of a previously-controlled disease or condition, in a subject who does not presently have, but is at risk of or is susceptible to, redeveloping a disease or condition or a recurrence of the disease or condition. Within the meaning of the invention, “treatment” therefore includes relapse prophylaxis or phase prophylaxis, as well as the treatment of acute or chronic signs, symptoms and/or malfunctions.

The term “treat” and synonyms contemplate administering a therapeutically effective amount of a compound of the invention to an individual in need of such treatment. The treatment can be orientated symptomatically, for example, to suppress symptoms. It can be effected over a short period, be oriented over a medium term, or can be a long-term treatment, for example within the context of a maintenance therapy.

The term “therapeutically effective amount” or “effective dose” as used herein refers to an amount of the active ingredient(s) that is (are) sufficient, when administered by a method of the invention, to efficaciously deliver the active ingredient(s) for the treatment of condition or disease of interest to an individual in need thereof.

The term “container” means any receptacle and closure therefor suitable for storing, shipping, dispensing, and/or handling a pharmaceutical product.

The term “insert” means information accompanying a pharmaceutical product that provides a description of how to administer the product, along with the safety and efficacy data required to allow the physician, pharmacist, and patient to make an informed decision regarding use of the product. The package insert generally is regarded as the “label” for a pharmaceutical product.

“Concurrent administration,” “administered in combination,” “simultaneous administration” and similar phrases mean that two or more agents are administered concurrently to the subject being treated. By “concurrently,” it is meant that each agent is administered simultaneously or sequentially in any order at different points in time. However, if not administered simultaneously, it is meant that they are administered to an individual in a sequence and within a time interval such that they can act in concert to provide an increased benefit than if they were administered otherwise.

For example, a benzolactam of structural formula (I) can be administered at the same time or sequentially in any order at different points in time as a second agent. However, if not administered at the same time, the agents should be administered sufficiently close in time so as to provide the desired therapeutic effect. A present benzolactam and the second agent can be administered separately, in any appropriate form and by any suitable route. When a present benzolactam and the second agent are not administered concurrently, it is understood that they can be administered in any order to a subject in need thereof. For example, a present benzolactam can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second agent, to an individual in need thereof. In various embodiments, a benzolactam of structural formula (I) and the second agent are administered 1 minute apart, 10 minutes apart, 30 minutes apart, less than 1 hour apart, 1 hour apart, 1 to 2 hours apart, 2 to 3 hours apart, 3 to 4 hours apart, 4 to 5 hours apart, 5 to 6 hours apart, 6 to 7 hours apart, 7 to 8 hours apart, 8 to 9 hours apart, 9 to 10 hours apart, 10 to 11 hours apart, 11 to 12 hours apart, no more than 24 hours apart or no more than 48 hours apart. In one embodiment, the components of the combination therapies are administered at 1 minute to 24 hours apart.

The use of the terms “a”, “an”, “the”, and similar referents in the context of describing the invention (especially in the context of the claims) are to be construed to cover both the singular and the plural, unless otherwise indicated. Recitation of ranges of values herein merely are intended to serve as a shorthand method of referring individually to each separate value falling within the range, unless otherwise indicated herein, and each separate value and subrange is incorporated into the specification as if it were individually recited herein. The use of any and all examples, or exemplary language (e.g., “such as”) provided herein, is intended to better illustrate the invention and is not a limitation on the scope of the invention unless otherwise claimed. No language in the specification should be construed as indicating any non-claimed element as essential to the practice of the invention.

In particular, the present invention is directed to benzolactams of structural formula (I), compositions comprising a compound of structural formula (I), and therapeutic uses of compounds of structural formula (I):

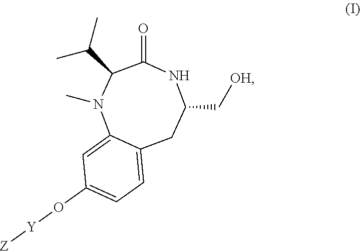

wherein Y is selected from the group consisting of null, C₁₋₈alkylene, NR^{b}, C(═O), C(═O)C₁₋₆alkylene, C₁₋₈alkyleneNR^{b}, C₁₋₄alkylenearyleneC₁₋₄alkylene, C₂₋₆alkenylene, C₄₋₈alkdienylene, C₁₋₆alkylenearylene, C(═O) arylene, C(═O)C₁₋₆alkylenearylene, C₁₋₆alkylenepiperidinyl, and C₂₋₆alkenylenearyleneC₁₋₄alkylene;
Z is selected from the group consisting of

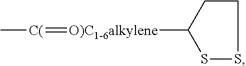

—C₃₋₁₀alkyleneSAc, —C(═O)N(R^{c})OH,

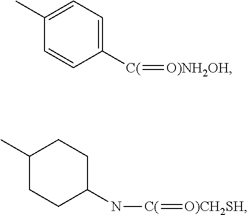

### —SO₂NHR^{c},

—NHSO₂NHR^{c}, —NHSO₂C₁₋₆alkyl, —SO₂C₁₋₆alkyl,

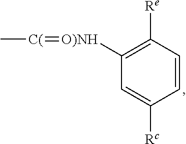

—C(═O)R^{f} wherein R^{f} is selected from the group consisting of OH, N(R^{c})², NH(OCH₃), N(CH₃)OH, C₁₋₆alkyl, CF₃, aryl, heteroaryl, C₃₋₈cycloalkyl, heterocycloalkyl, NHSO₂CH₃, NHSO₂CF₃, and C₁₋₆haloalkyl, —C(═O)(C(R^{c})²)₁₋₃SH, —SR^{d} wherein R^{d} is hydrogen or (C═O)CH₃,

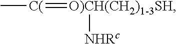

—S—(C═O)C₁₋₆alkyl, heterocycloalkyl optionally substituted with oxo (═O), thioxo (═S), or both, heteroaryl optionally substituted with —NH₂, —SH, or both, —N(H)C(═O)SH, —NHC(═O)NHR^{e}, —NHC(═O)CH₂R^{e}, —NHC(═O)(CH₂)₁₋₆SH, —NHC(═O)CH₂Hal, —NHC(═S)NHR^{e}, —NHC(═S)CH₂R^{e}, —C(═S)NHR^{c}, —C(═S)CH₂R^{e}, —NHC(═S)CH₂R^{e}, —NHC(═S)CH₂Hal, and —(C═O)C₁₋₃alkyl;
R^{b}, independently, is selected from the group consisting of hydrogen, C₁₋₆alkyl, aryl, heteroaryl, C₃₋₈cycloalkyl, and heterocycloalkyl;
R^{c}, independently, is selected from the group consisting of hydrogen, (C═O)CH₃, C₁₋₆alkyl, CF₃, CH₂F, and aryl, or two R^{c} groups are taken together with the carbon to which they attached to form a C₃₋₈cycloalkyl group; and
R^{e} is NH₂ or OH;
or a pharmaceutically acceptable salt, hydrate, or prodrug thereof.

The compounds of structural formula (I) activate PKC and inhibit HDAC, and are useful in the treatment of a variety of diseases and conditions. In particular, benzolactams of structural formula (I) are used in methods of treating a disease or condition wherein activation of PKC and inhibition of HDAC provides a benefit, for example, neurological diseases. The methods comprise administering a therapeutically effective amount of a benzolactam of structural formula (I) to an individual in need thereof.

The present methods also encompass administering a second therapeutic agent to the individual in addition to a benzolactam of structural formula (I). The second therapeutic agent is selected from agents, such as drugs and adjuvants, known as useful in treating the disease or condition afflicting the individual, e.g., a therapeutic agent known as useful in treating Alzheimer's Disease.

As used herein, the term “alkyl” refers to straight chained and branched saturated hydrocarbon groups, nonlimiting examples of which include methyl, ethyl, and straight chain and branched propyl, butyl, pentyl, hexyl, heptyl, and octyl groups containing the indicated number of carbon atoms. The term Cₙ means the alkyl group has “n” carbon atoms.

The term “alkylene” refers to a bidentate moiety obtained by removing two hydrogen atoms from an alkane. An “alkylene” is positioned between two other chemical groups and serves to connect them. An example of an alkylene group is —(CH₂)ₙ—. An alkyl, e.g., methyl, or alkylene, e.g., —CH₂CH₂—, group can be substituted with halo, trifluoromethyl, trifluoromethoxy, hydroxy, alkoxy, nitro, cyano, alkylamino, or amino groups, for example.

The term “alkenyl” is defined identically as “alkyl,” except for containing a carbon-carbon double bond, e.g., ethenyl, propenyl, and butenyl. The term “alkenylene” is defined identically to “alkylene” except for containing a carbon-carbon double bond. The term “alkdienylene” is defined identically as “alkenylene” except the group contains two carbon-carbon double bonds, either conjugated or non-conjugated.

As used herein, the term “halo” and “Hal” are defined as fluoro, chloro, bromo, and iodo.

The term “hydroxy” is defined as —OH.

The term “alkoxy” is defined as —OR, wherein R is alkyl.

The term “amino” is defined as —NR₂, wherein each R group, independently, is hydrogen, alkyl, cycloalkyl, C₁₋₃alkylenearyl, heteroaryl, or aryl, or both R groups are taken together with the N to which they are attached to form a 4 to 8 membered ring.

The term “nitro” is defined as —NO₂.

The term “cyano” is defined as —CN.

The term “trifluoromethyl” is defined as —CF₃.

The term “trifluoromethoxy” is defined as —OCF₃.

The term “Ac” is defined as —C(═O)CH₃.

The term “SAc” is defined as C(═S)CH₃.

The term “tBu” is defined as tertiary butyl, i.e. —C(CH₃)₃.

As used herein, compounds such as

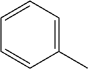

is an abbreviation for

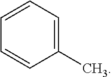

As used herein, groups such as C₁₋₃alkylphenyl means a C₁₋₃alkyl group bonded to a phenyl ring, for example,

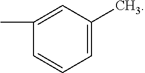

Groups such as C₁₋₃alkylenephenyl means a phenyl group bonded to a C₁₋₃alkylene group, for example,

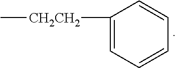

As used herein, the term “aryl” refers to a monocyclic aromatic group, e.g., phenyl. Unless otherwise indicated, an aryl group can be unsubstituted or substituted with one or more, and in particular one to five, groups independently selected from, for example, halo, alkyl, alkenyl, —OCF₃, NO₂, CN, NC, OH, alkoxy, amino, alkylamino, —CO₂H, —CO₂alkyl, aryl, and heteroaryl. Exemplary aryl groups include, but are not limited to, phenyl, chlorophenyl, methylphenyl, methoxyphenyl, trifluoromethylphenyl, nitrophenyl, 2,4-methoxychlorophenyl, and the like.

The term “arylene” refers to a bidentate aryl group that bonds to two other groups and serves to connect these groups e.g.,

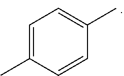

The term “C₁₋₄alkylenearyleneC₁₋₄alkylene” means

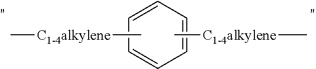

and serves to connect two other groups.

The term “C₁₋₆alkylenearylene” means

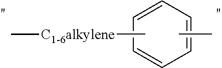

and serves to connect two other groups.

The term “C₂₋₆alkenylenearyleneC₁₋₄alkylene” means

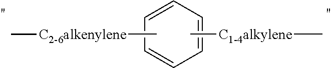

and serves to connect two other groups.

As used herein, the term “heteroaryl” refers to a monocyclic ring system containing at least one nitrogen, oxygen, or sulfur atom in an aromatic ring. Unless otherwise indicated, a heteroaryl group can be unsubstituted or substituted with one or more, and in particular one to four, substituents selected from, for example, halo, alkyl, alkenyl, —OCF₃, —NO₂, —CN, —NC, —OH, alkoxy, amino, alkylamino, —CO₂H, —CO₂alkyl, aryl, and heteroaryl. Examples of heteroaryl groups include, but are not limited to, thienyl, furyl, oxazolyl, thiophenyl, triazolyl, isothiazolyl, isoxazolyl, imidazolyl, pyrimidinyl, thiazolyl, thiadiazolyl, pyridinyl, pyridazinyl, pyrazolyl, pyrazinyl, tetrazolyl, oxazolyl, pyrrolyl, and triazinyl.

As used herein, the term “C₃₋₈cycloalkyl” means a monocyclic aliphatic ring containing three to eight carbon atoms, either saturated or unsaturated.

As used herein, the term “heterocycloalkyl” means a monocyclic or a bicyclic aliphatic ring containing 5 to 10 total atoms, either saturated or unsaturated, of which one to five of the atoms are independently selected from nitrogen, oxygen, and sulfur and the remaining atoms are carbon.

In some embodiments, Y is null,

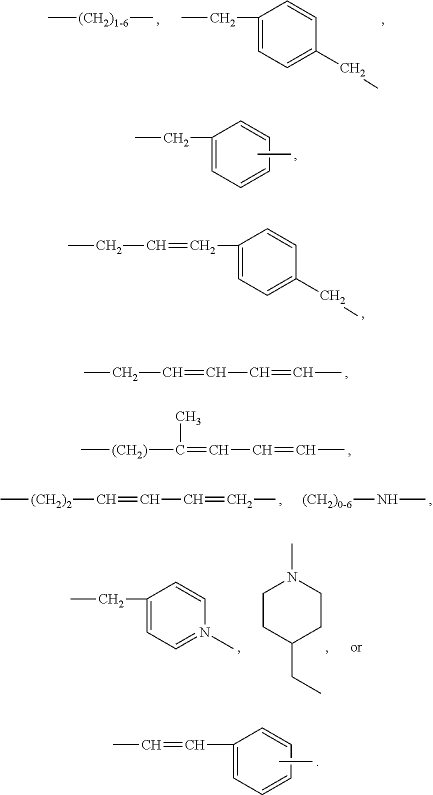

In still other embodiments, Z is

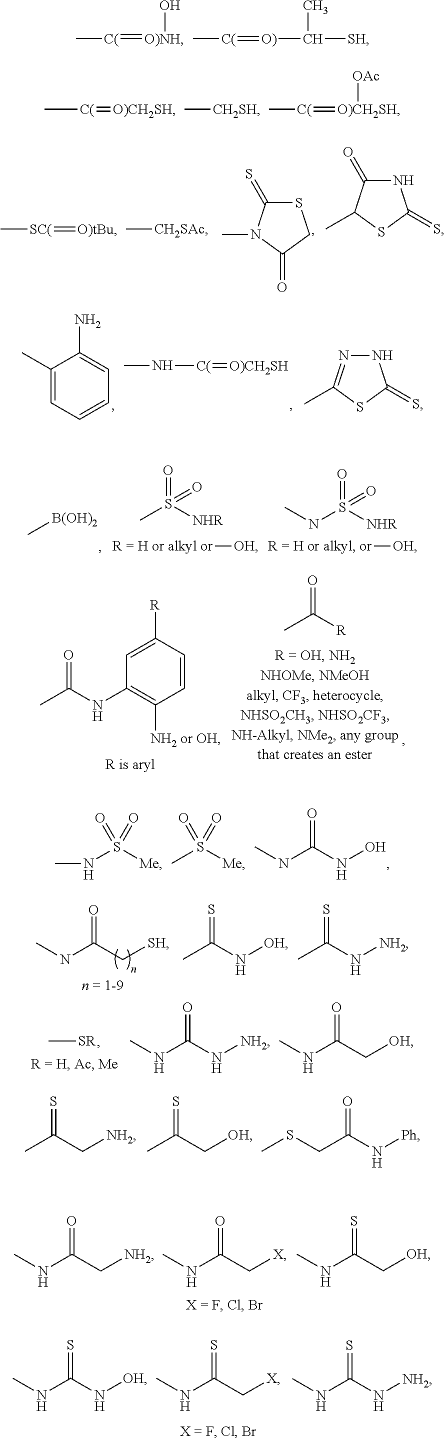

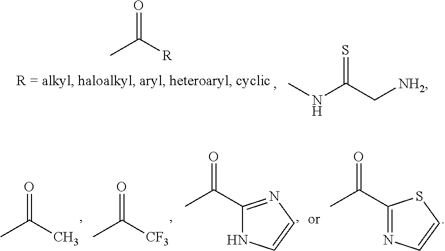

Additionally, salts, prodrugs, and hydrates of the present benzolactams also are included in the present invention and can be used in the methods disclosed herein. The present invention further includes all possible stereoisomers and geometric isomers of the compounds of structural formula (I). The present invention includes both racemic compounds and optically active isomers. When a benzolactam of structural formula (I) is desired as a single enantiomer, it can be obtained either by resolution of the final product or by stereospecific synthesis from either isomerically pure starting material or use of a chiral auxiliary reagent, for example, see Z. Ma et al., Tetrahedron: Asymmetty, 8(6), pages 883-888 (1997). Resolution of the final product, an intermediate, or a starting material can be achieved by any suitable method known in the art. Additionally, in situations where tautomers of the compounds of structural formula (I) are possible, the present invention is intended to include all tautomeric forms of the compounds.

Prodrugs of compounds of structural formula (I) also are included in the present invention. It is well established that a prodrug approach, wherein a compound is derivatized into a form suitable for formulation and/or administration, then released as a drug in vivo, has been successfully employed to transiently (e.g., bioreversibly) alter the physicochemical properties of the compound (see, H. Bundgaard, Ed., “Design of Prodrugs,” Elsevier, Amsterdam, (1985); R. B. Silverman, “The Organic Chemistry of Drug Design and Drug Action,” Academic Press, San Diego, chapter 8, (1992); K. M. Hillgren et al., Med. Res. Rev., 15, 83 (1995)). Specific prodrugs of HDAC[s are discussed in WO 2008/055068, incorporated in its entirety herein by reference.

Compounds of the present invention can contain one or more functional groups. The functional groups, if desired or necessary, can be modified to provide a prodrug. Suitable prodrugs include, for example, acid derivatives, such as amides and esters. It also is appreciated by those skilled in the art that N-oxides can be used as a prodrug.

Compounds of the invention can exist as salts. Pharmaceutically acceptable salts of the present benzolactams often are preferred in the methods of the invention. As used herein, the term “pharmaceutically acceptable salts” refers to salts or zwitterionic forms of the compounds of structural formula (I). Salts of compounds of formula (I) can be prepared during the final isolation and purification of the compounds or separately by reacting the compound with an acid having a suitable cation. The pharmaceutically acceptable salts of compounds of structural formula (I) can be acid addition salts formed with pharmaceutically acceptable acids. Examples of acids which can be employed to form pharmaceutically acceptable salts include inorganic acids such as nitric, boric, hydrochloric, hydrobromic, sulfuric, and phosphoric, and organic acids such as oxalic, maleic, succinic, tartaric, and citric. Nonlimiting examples of salts of compounds of the invention include, but are not limited to, the hydrochloride, hydrobromide, hydroiodide, sulfate, bisulfate, 2-hydroxyethansulfonate, phosphate, hydrogen phosphate, acetate, adipate, alginate, aspartate, benzoate, bisulfate, butyrate, camphorate, camphorsulfonate, digluconate, glycerolphosphate, hemisulfate, heptanoate, hexanoate, formate, succinate, fumarate, maleate, ascorbate, isethionate, salicylate, methanesulfonate, mesitylenesulfonate, naphthylenesulfonate, nicotinate, 2-naphthalenesulfonate, oxalate, pamoate, pectinate, persulfate, 3-phenylproprionate, picrate, pivalate, propionate, trichloroacetate, trifluoroacetate, phosphate, glutamate, bicarbonate, paratoluenesulfonate, undecanoate, lactate, citrate, tartrate, gluconate, methanesulfonate, ethanedisulfonate, benzene sulphonate, and p-toluenesulfonate salts. In addition, available amino groups present in the compounds of the invention can be quaternized with methyl, ethyl, propyl, and butyl chlorides, bromides, and iodides; dimethyl, diethyl, dibutyl, and diamyl sulfates; decyl, lauryl, myristyl, and stearyl chlorides, bromides, and iodides; and benzyl and phenethyl bromides. In light of the foregoing, any reference to compounds of the present invention appearing herein is intended to include compounds of structural formula (I) as well as pharmaceutically acceptable salts, hydrates, or prodrugs thereof.

The compounds of structural formula (I) also can be conjugated or linked to auxiliary moieties that promote a beneficial property of the compound in a method of therapeutic use. Such conjugates can enhance delivery of the compounds to a particular anatomical site or region of interest (e.g., a tumor), enable sustained therapeutic concentrations of the compounds in target cells, alter pharmacokinetic and pharmacodynamic properties of the compounds, and/or improve the therapeutic index or safety profile of the compounds. Suitable auxiliary moieties include, for example, amino acids, oligopeptides, or polypeptides, e.g., antibodies, such as monoclonal antibodies and other engineered antibodies; and natural or synthetic ligands to receptors in target cells or tissues. Other suitable auxiliaries include fatty acid or lipid moieties that promote biodistribution and/or uptake of the compound by target cells (see, e.g., Bradley et al., Clin. Cancer Res. (2001) 7:3229).

Specific compounds of the present invention include, but are not limited to,

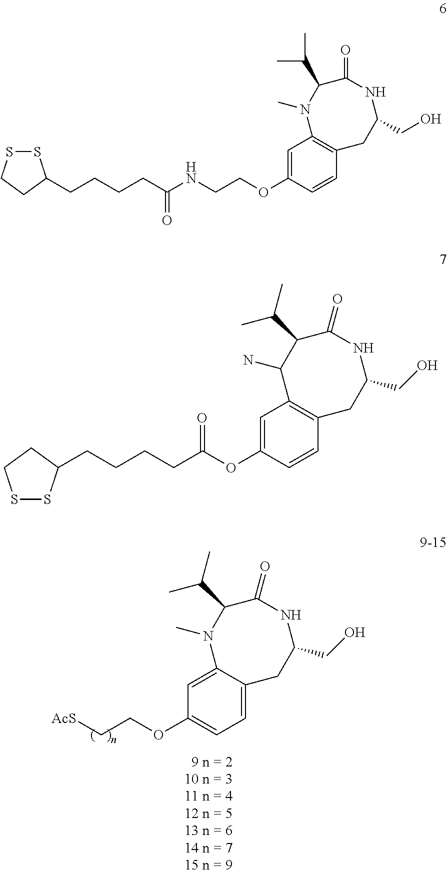

The following synthetic schemes are representative of the reactions used to synthesize compounds of structural formula (I). Modifications and alternate schemes to prepare benzolactams of the invention are readily within the capabilities of persons skilled in the art.

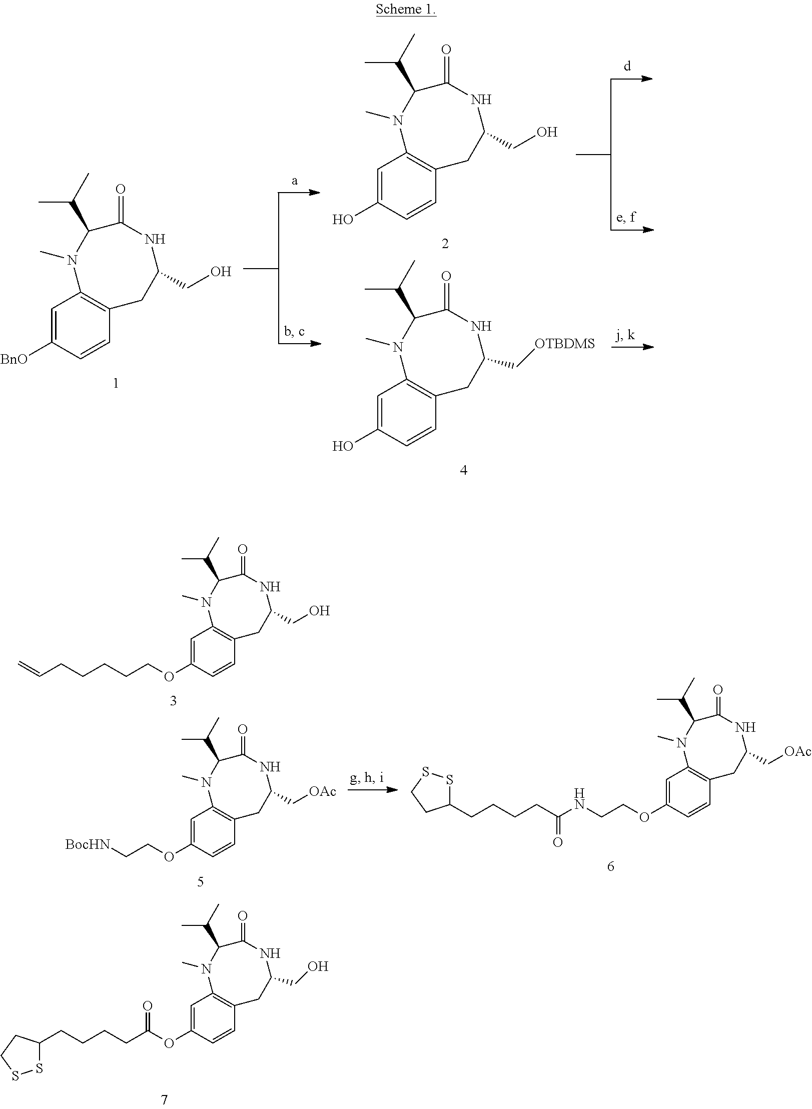

Reagent and Conditions (a) H₂, 10% Pd/C, EtOAc; (b) TBDMS-Cl, Et₃N, DMAP, CH₂Cl₂, 0° C. to rt (room temperature), 12 h; (c) H₂, 20% Pd(OH)₂/C, EtOAc, rt, 1 h; (d) 7-bromo-1-heptene, K₂CO₃, KI, DMF, 80° C;. (e) BocNHCH₂CH₂Br, Cs₂CO₃, 100° C., 24 h; (f) Ac₂O, pyridine, CH₂Cl₂, 0° C. to rt, 20 h; (g) TFA, CH₂Cl₂, 0° C. to rt, 2 h; (h) (±)-α-Lipoic acid, EDCI, HOBT, DIEA, DMF, 0° C. to rt, 12 h; (i) K₂CO₃, MeOH, 0° C., 30 min; (i) (±)-α-Lipoic acid, DCC, DMAP, CH₂Cl₂, rt, 16 h;. (k) PTSA, MeOH, rt, 3 h.

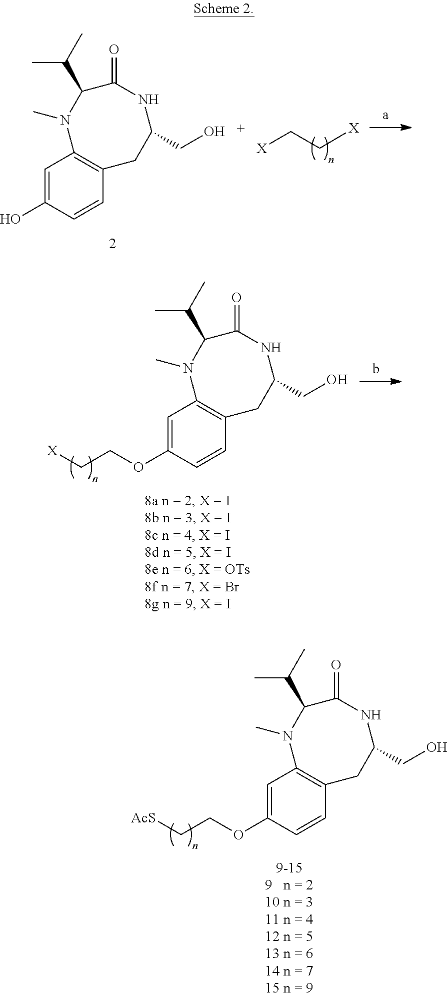

Reagents and Conditions for compounds 9-15: (a) Cs₂CO₃ , DMF, 80° C., 8 h; (b) AcSK, DMF, 50° C., 1 h.

As shown above, compounds of the invention were synthesized by starting from the common intermediate 1 [28], which was subjected to benzyl deprotection with hydrogen (H₂) to obtain the phenol compound 2 (Scheme 1). Next, compound 2 was treated with 7-bromo-1-heptene in the presence of potassium carbonate (K₂CO₃) as a base and potassium iodide (KI) at 80° C. for 7 hours to afford compound 3 in 31% yield. Compound 5 was synthesized in 16% yield from compound 2 using two steps: (a) treatment with Boc-protected 2-bromoethanolamine (BocNHCH₂CH₂Br) in the presence of cesium carbonate (CS₂CO₃) to give the intermediate phenolic ether in 20% yield, followed by (b) acetylation of the free hydroxy group using acetic anhydride and pyridine as a base.

The synthesis of compound 6 was carried out by starting from the Boc-protected amino compound 5 and coupling it with (±)-α-lipoic acid using EDCI and HOBT followed by deacetylation under basic conditions, giving 80% yield. Compound 7 was prepared from compound 1 by protection of the free hydroxy group with TBDMSCl, followed by debenzylation in the usual way to afford compound 4. Compound 4 was allowed to react with (±)-α-lipoic acid using DCC as the coupling reagent followed by silyl deprotection to give the final product in 36% yield. Compounds 9-15 also were derived from compound 2, which was coupled with the appropriate disubstituted alkanes to provide the intermediate compound 8 (Scheme 2). Compound 8 then was treated with potassium thioacetate at 50° C. for 2 hours to afford the compounds 9-15 in 12-67% yield, as shown in Scheme 2.

¹H NMR and ¹³C NMR spectra were recorded on a Bruker spectrometer at 300/400 MHz and 75/100 MHz, respectively, with (CH₃)₄Si (tetramethylsilane) as an internal standard. HRMS experiments were performed on a QTOF-2TM instrument (Micromass). TLC was performed with Merck 60 F₂₅₄ silica gel plates. Preparative TLC was performed with Analtech 1000 mm silica gel GF plates. Column chromatography was performed with Merck silica gel (40-60 mesh). HPLC was carried out on an ACE AQ column (100×4.6 mm and 250×10 mm) with detection at 210, 240, 254, 280, and 300 nm on a Shimadzu SPD-10A VP detector; flow rate=2.0-3.5 mLmin⁻¹, from 10% CH₃CN (acetonitrile) in H₂O to 100% CH₃CN with 0.05% trifluoroacetic acid (TFA).

9-Hydroxy-5-hydroxymethyl-2-isopropyl-1-methyl-(2S,5S)-1,4,5,6-tetrahydro-2H-benzo[e][1,4]diazocin-3-one (2): Compound 1 (500 mg, 1.36 mmol) was dissolved in EtOAc (ethyl acetate) (20 mL) and subjected to debenzylation with H₂ (hydrogen gas) in the presence of 10% Pd/C (palladium on carbon) as catalyst. After 30 min, the reaction mixture was filtered, the filtrate was concentrated, and purified by column chromatography to afford compound 2 (288 mg, 76%); [α]_{D}²⁰=−331.18 (c=0.10 in MeOH); ¹H NMR (300 MHz, CD₃OD): δ=6.86 (d, J=8.3 Hz, 1H), 6.55 (d, J=2.2 Hz, 1H), 6.37 (dd, J=2.3, 8.2 Hz, 1H), 4.28 (m, 1H), 3.59 (dd, J=4.8, 11.0 Hz, 1H), 3.47 (m, 2H), 2.86 (m, 2H), 2.73 (s, 3H), 2.38 (m, 1H), 1.08 (d, J=6.7 Hz, 3H), 0.93 ppm (d, J=6.7 Hz, 3H); ¹³C NMR (100 MHz, CD₃OD): δ=175.9, 158.3, 154.4, 133.6, 124.8, 111.2, 108.9, 65.9, 55.6, 37.9, 37.0, 29.8, 20.9, 20.1 ppm; ESI-HRMS calculated for [C₁₅H₂₃N₂O₃+H]⁺: 279.1703, found: 279.1693; HPLC purity: 96.2%.

9-Hept-6-enyloxy-5-hydroxymethyl-2-isopropyl-1-methyl-1,4,5,6-tetrahydro-2H-benzo[e][1,4]diazocin-3-one (3): 7-Bromo-l-heptene (44 μL, 0.29 mmol), KI (72 mg, 0.43 mmol), and K₂CO₃ (80 mg, 0.58 mmol) were added to a solution of compound 2 (40 mg, 0.14 mmol) in anhydrous N,N-dimethylformamide (DMF; 2 mL), and then heated at 80° C. for 8 h. The mixture was cooled and extracted with EtOAc (50 mL), washed with H₂O (30 mL) and brine (30 mL), dried over anhydrous Na₂SO₄ (sodium sulfate), concentrated, and purified by column chromatography to afford compound 3 (17 mg, 31%); [α]_{D}²⁰=−221.23 (c=0.09 in MeOH); ¹H NMR (400 MHz, CDCl₃): δ=6.93 (brs, 1H), 6.92 (d, J=8.0 Hz, 1H), 6.52 (d, J=4.0 Hz, 1H), 6.41 (dd, J=4.0, 8.0 Hz, 1H), 5.87-5.77 (m, 1H), 5.03-4.94 (m, 2H), 3.93-3.89 (m, 3H), 3.72-3.66 (m, 2H), 3.50-3.48 (m, 2H), 3.03 (dd, J=8.0, 16.0 Hz, 1H), 2.77 (s, 3H), 2.77-2.69 (m, 1H), 2.45-2.37 (m, 1H), 2.09-2.08 (m, 2H), 1.79-1.76 (m, 2H), 1.47-1.46 (m, 4H), 1.05 (d, J=8.0 Hz, 3H), 0.87 ppm (d, J=8.0 Hz, 3H); ¹³C NMR (100 MHz, CDCl₃): δ=174.4, 159.0, 152.8, 139.0, 132.5, 123.2, 114.7, 106.8, 106.6, 70.0, 68.1, 66.1, 54.8, 36.7, 35.3, 33.9, 29.4, 28.9, 28.5, 25.8, 20.7, 20.1 ppm; ESI-HRMS calculated for [C₂₂H₃₄N₂O₃+H]⁺: 375.2648, found: 375.2642; HPLC purity: 95.1%.

5-(O-tert-Butyldimethylsilanyloxymethyl)-9-hydroxy-2-isopropyl-1-methyl-(2S,5S)-1,4,5,6-tetrahydrodro-2H-benzo[e][1,4]diazocin-3-one (4): Et₃N (triethylamine) (2.30 mL, 16.50 mmol) and tert-butyldimethylsilyl chloride (TBDMSCl; 1.60 g, 10.62 mmol) were added to a solution of compound 1 (1.00 g, 2.71 mmol) in anhydrous CH₂Cl₂ (methylene chloride) (20 mL) at 0° C., the mixture was stirred for another 5 min followed by the addition of N,N-dimethyl-4-aminopyridine (DMAP; 66 mg, 0.54 mmol), and then stirred for an additional 3 h. The reaction mixture was quenched by a saturated solution of NH₄Cl (aq) (ammonium chloride), and then extracted with CH₂Cl₂ (80 mL), washed with a solution of HCl (aq) (hydrochloric acid) (0.5 N, 10 mL), H₂O (40 mL), and brine (40 mL), dried over anhydrous Na₂SO₄, and concentrated to afford the crude residue, which was dissolved in EtOAc (20 mL) and subjected to debenzylation with H₂ under balloon pressure in the presence of 20% Pd(OH)₂/C (Pearlman's catalyst) as catalyst. The reaction mixture was filtered after 30 min, and the filtrate was concentrated and purified by column chromatography to afford compound 4 (806 mg, 76%); [α]_{D}²⁰=−232.51 (c=0.11 in MeOH); ¹H NMR (400 MHz, CDCl₃): δ=6.82 (d, J=8.1 Hz, 1H), 6.50 (s, 1H), 6.30 (dd, J=2.4, 8.2 Hz, 1H), 6.12 (d, J=3.2 Hz, 1H), 6.06 (s, 1H), 4.06 (m, 1H), 3.64 (dd, J=3.9, 9.8 Hz, 1H), 3.50 (dd, J=2.4, 15.3 Hz, 2H), 2.98 (dd, J=8.0, 16.4 Hz, 1H), 2.75 (s, 3H), 2.68 (dd, J=8.0, 16.4 Hz, 1H), 2.42 (m, 1H), 1.06 (d, J=7.0 Hz, 3H), 0.91 (s, 9H), 0.89 (d, J=7.0 Hz, 3H), 0.09 (s, 3H), 0.07 ppm (s, 3H); ¹³C NMR (100 MHz, CDCl₃): δ=173.3, 155.8, 152.8, 132.4, 122.7, 108.9, 107.0, 71.3, 65.9, 53.8, 36.7, 35.5, 28.3, 25.9, 19.9, 18.3, −5.3, −5.4 ppm; ESI-HRMS calculated for [C₂₁H₃₆N₂O3Si+H]⁺: 393.2568, found: 393.2572.

2-Isopropyl-1-methyl-9-(2-tert-butyloxycarboxamidoethoxy)-3-oxo-(2S,5S)-1,2,3,4,5,6-hexahydrobenzo[e][1,4]diazocin-5-ylmethyl acetate (5): 2-tert-Boc-aminoethylbromide (805 mg, 3.61 mmol) and Cs₂CO₃ (1.80 g, 5.52 mmol) were added to a solution of compound 2 (500 mg, 1.81 mmol) in anhydrous DMF (10 mL), and the mixture was heated at 100° C. for 24 h. The mixture was extracted with EtOAc (200 mL), washed with H₂O (100 mL) and brine (100 mL), dried over anhydrous Na₂SO4, concentrated, and purified by column chromatography to afford the intermediate alcohol (152 mg, 20%); [α]_{D}²⁰=−189.10 (c=0.10 in MeOH); ¹H NMR (400 MHz, CDCl₃): δ=6.94 (d, J=9.0 Hz, 1H), 6.68 (brs, 1H), 6.51 (d, J=2.0 Hz, 1H), 6.39 (dd, J=2.0, 9.0 Hz, 1H), 5.01 (brs, 1H), 3.97 (dd, J=5.0, 10.0 Hz, 2H), 3.88 (brs, 1H), 3.68 (m, 2H), 3.49 (m, 4H), 3.04 (dd, J=7.8, 16.5 Hz, 1H), 2.77 (s, 3H), 2.69 (dd, J=7.8, 16.5 Hz, 1H), 2.41 (m, 1H), 1.45 (s, 9H), 1.05 (d, J=7.0 Hz, 3H), 0.86 ppm (d, J=7.0 Hz, 3H); ¹³C NMR (75 MHz, CDCl₃): δ=173.7, 158.5, 156.2, 152.9, 132.6, 123.7, 106.7, 106.4, 79.8, 67.3, 66.0, 54.8, 40.4, 36.8, 35.2, 29.9, 28.6, 28.5, 20.7, 20.1 ppm; ESI-HRMS calculated for [C₂₂H₃₆N₃O₅+H]⁺: 422.2649, found: 422.2638.

The intermediate alcohol (200 mg, 0.47 mmol) was dissolved in anhydrous CH₂Cl₂ (3 mL) and cooled to 0° C. Pyridine (0.23 mL, 2.82 mmol) and Ac₂O (acetic anhydride) (0.20 mL, 2.12 mmol) were added sequentially, and the reaction mixture was stirred at room temperature for 12 h. The reaction mixture was extracted with EtOAc (60 mL), washed with a solution of HCl (aq) (1 N, 10 mL), H₂O (20 mL), and brine (20 mL), dried over anhydrous Na₂SO₄, concentrated, and purified by column chromatography to compound afford compound 5 (170 mg, 78%); ¹H NMR (400 MHz, CDCl₃): δ=6.89 (d, J=8.1 Hz, 1H), 6.54 (s, 1H), 6.40 (d, J=8.1 Hz, 1H), 5.93 (s, 1H), 4.99 (brs, 1H), 4.32 (m, 1H), 4.15 (dd, J=3.4, 11.0 Hz, 1H), 3.92 (m, 3H), 3.44 (m, 3H), 2.89 (m,2H), 2.73 (s, 3H), 2.37 (m, 1H), 2.06 (s, 3H), 1.41 (s, 9H), 1.02 (d, J=6.4 Hz, 3H), 0.87 ppm (d, J=6.4 Hz, 3H); ¹³C NMR (100 MHz, CDCl₃): δ=173.1, 170.8, 158.6, 156.0, 152.9, 132.6, 123.4, 107.7, 107.1, 79.7, 71.7, 67.3, 67.0, 51.7, 40.3, 37.3, 35.9, 28.5, 21.0, 20.2, 19.9 ppm; ESI-HRMS calculated for [C₂₄H₃₇N₃O₆+Na]⁺: 486.2575, found: 486.2567.

1N-{2-[5-Hydroxymethyl-2-isopropyl-1-methyl-3-oxo-(2S,5S)-1,2,3,4,5,6-hexahydrobenzo[e][1,4]diazocin-9-yloxy]ethyl}-5-(1,2-dithiolan-3-yl)pentanamide (6): TFA (trifluoroacetic acid) (0.4 mL) was added to a solution of compound 5 (63 mg, 0.14 mmol) in anhydrous CH₂Cl₂ (1.6 mL) at 0° C., and the reaction mixture was stirred at room temperature for 3 h. Solvent and excess TFA were removed under reduced pressure, and the crude residue was dried under high vacuum and subjected to the next reaction without further purification.

1-Hydroxy-1H-benzotriazole (HOBT; 16 mg, 0.12 mmol) and N′-(3-dimethylaminopropyl)-N-ethylcarbodiimide (EDCl; 24 mg, 0.12 mmol) were added to a solution of (±)-α-lipoic acid (25 mg, 0.12 mmol) in anhydrous CH₂Cl₂ (2 mL) at 0° C. and stirred for 15 min. The crude amine dissolved in anhydrous CH₂Cl₂ (2 mL, free amine was generated from TFA amine salt by adding N,N-diisopropylethylamine (DIEA; 0.1 mL) to a CH₂Cl₂ solution of TFA salt) was added to the active ester solution, and the resultant mixture was stirred at room temperature for 12 h. The reaction was quenched by adding a saturated solution of NH₄Cl (aq) (10 mL) and extracted with EtOAc (40 mL), washed with H₂O (20 L), and brine (20 mL), dried over anhydrous Na₂SO₄, and concentrated to give the crude residue. The residue was dissolved in anhydrous MeOH (methanol) (2 L) and cooled to 0° C.; K₂CO₃ (16 mg, 0.12 mmol) was added, and the mixture was stirred for another 30 min at the same temperature. The mixture was extracted with EtOAc (50 mL), washed with H₂O (25 mL) and brine (25 mL), dried over anhydrous Na₂SO₄, concentrated, and purified by column chromatography to afford compound 6 (49 mg, 80%); [α]_{D}²⁰=−136.44 (c=0.11 in MeOH); ¹H NMR (400 MHz, CDCl₃): δ=6.93 (d, J=8.0 Hz, 1H), 6.84 (s, 1H), 6.52 (s, 1H), 6.40 (d, J=8.0 Hz, 1H), 6.11 (t, J=6.0 Hz, 1H), 4.0 (m, 2H), 3.86 (m, 2H), 3.59 (m, 3H), 3.48 (m, 3H), 3.12 (m, 3H), 2.76 (s, 3H), 2.75 (m, 1H), 2.42 (m, 2H), 2.22 (t, J=8.0 Hz, 2H), 1.84 (m, 1H), 1.66 (m, 4H), 1.46 (m, 2H), 1.05 (t, J=7.0 Hz, 3H), 0.87 ppm (t, J=7.0 Hz, 3H); ¹³C NMR (100 MHz, CDCl₃): δ=173.8, 173.0, 158.1, 152.7, 132.5, 123.8, 106.7, 106.2, 70.2, 66.7, 65.7, 56.3, 54.4, 40.2, 38.9, 38.4, 36.5, 36.3, 35.2, 34.5, 28.8, 28.2, 22.6, 20.3, 19.9 ppm; ESIHRMS calculated for [C₂₃H₃₉N₃O₄S₂+Na]⁺: 532.2275, found: 532.2262. HPLC purity: 98.6%.

(2S,5S)-1,2,3,4,5,6-Hexahydro-5-(hydroxymethyl)-2-isopropyl-1-methyl-3-oxobenzo[e][1,4]diazocin-9-yl-5-(1,2-dithiolan-3-yl)pentanoate (7): N,N′-Dicyclohexylcarbodiimide (DCC; 33 mg, 0.16 mmol) and N,N-dimethyl-4-aminopyridine (9 mg, 0.07 mmol) were added to a solution of (±)-α-lipoic acid (30 mg, 0.15 mmol) in CH₂Cl₂ (2 mL) at 0° C. A solution of compound 4 (68 mg, 0.17 mmol, dissolved in 1 mL CH₂Cl₂) then was added. The reaction mixture was warmed to room temperature and stirred for another 3 h, then extracted with EtOAc (40 mL), washed with a saturated solution of NaHCO₃(aq) (sodium bicarbonate) (10 mL), H₂O (20 mL), and brine (20 mL), dried over anhydrous Na₂SO₄, concentrated, and purified by column chromatography to yield the ester intermediate. The ester was subjected to TBDMS (t-butyldimethylsiloxy) deprotection with p-toluenesulfonic acid (28 mg, 0.15 mmol) in MeOH for 3 h at room temperature, then extracted with EtOAc (40 mL), washed with H₂O (20 mL) and brine (20 mL), dried over anhydrous Na₂SO₄, concentrated and purified by column chromatography to afford compound 7 (25 mg, 36%); [α]_{D}²⁰=−235.88 (c=0.17 in MeOH); ¹NMR (400 MHz, CDCl₃); δ=7.03 (d, J=8.3 Hz, 1H), 6.78 (s, 1H), 6.68 (d, J=2.2 Hz, 1H), 6.60 (dd, J=2.2, 8.3 Hz, 1H), 3.81 (m, 1H), 3.66 (m, 1H), 3.47 (m, 3H), 3.12 (m, 3H), 2.77 (s, 3H), 2.74 (m, 1H), 2.53 (m, 2H), 2.43 (m, 2H), 1.76 (m, 1H), 1.72 (m, 4H), 1.53 (m, 3H), 1.03 (d, J=6.4 Hz, 3H), 0.83 ppm (d, J=6.4 Hz, 3H); ¹³C NMR (100 MHz, CDCl₃): δ=173.9, 172.2, 152.6, 150.4, 132.6, 128.2, 114.3, 112.7, 69.7, 66.2, 56.6, 54.4, 40.5, 38.8, 37.0, 35.1, 34.8, 34.4, 28.9, 28.4, 24.8, 20.8, 20.0 ppm; ESI-HRMS calculated for [C₂₃H₃₄N₂O₄S₂+H]: 467.2033, found: 467.2032; HPLC purity: 96.3%.

Thioacetic acid S-[3-(5-hydroxymethyl-2-isopropyl-1-methyl-3-oxo-1,2,3,4,5,6-hexahydrobenzole[e][1,4]diazocin-9-yloxy)propyl]ester (9): 1,3-Diiodopropane (207 μL, 1.80 mmol) and Cs₂CO₃ (234 mg, 0.72 mmol) were added to a solution of compound 2 (50 mg, 0.18 mmol) in anhydrous DMF (2 mL), and the mixture was heated at 80° C. for 8 h. The mixture was cooled and then extracted with EtOAc (30 mL), washed with H₂O (15 mL) and brine (15 mL), dried over anhydrous Na₂SO₄, concentrated, and purified by column chromatography to afford the iodo-substituted intermediate (20 mg, 0.037 mmol). The intermediate was dissolved in anhydrous DMF (0.5 mL), and potassium thioacetate (AcSK) (20 mg, 0.19 mmol) was added to the solution. The mixture then was heated at 50° C. for 1 h. The mixture was extracted with EtOAc (30 mL), washed with H₂O (15 mL) and brine (15 L), dried over anhydrous Na₂SO₄, concentrated, and purified by column chromatography to afford compound 9 (6 mg, 12%); [α]_{D}²⁰−=232.12 (c=0.075 in MeOH); ¹H NMR (400 MHz, CDCl₃): δ=6.94 (d, J=8.0 Hz, 1H), 6.85 (brs, 1H), 6.54 (d, J=2.0 Hz, 1H), 6.43 (dd, J=2.0, 8.0 Hz, 1H), 4.01 (brs, 1H), 3.98 (t, J=8.0 Hz, 2H), 3.74 (dd, J=4.0. 12.0 Hz, 1H), 3.59-3.51 (m, 2H), 3.07-3.01 (m, 3H), 2.80 (s, 3H), 2.80-2.76 (m, 1H), 2.48-2.41 (m, 1H), 2.34 (s, 3H), 2.08-2.02 (m, 2H), 1.06 (d, J=8.0 Hz, 3H), 0.89 ppm (d, J=8.0 Hz, 3H); ¹³C NMR (100 MHz, CDCl₃): δ=195.8, 174.4, 158.6, 152.6, 132.4, 123.2, 107.0, 106.7, 70.3, 66.2, 65.9, 54.6, 36.5, 35.3, 30.7, 29.4, 28.3, 25.9, 20.3, 19.9 ppm; ESI-HRMS calculated for [C₂₀H₃₀N₂O₄S+H]⁺: 395.2005, found 395.2006; HPLC purity: 96.4%.

Thioacetic acid S-[4-(5-hydroxymethyl-2-isopropyl-1-methyl-3-oxo-1,2,3,4,5,6-hexahydrobenzo[e][1,4]diazocin-9-yloxy)butyl] ester (10): Compound 10 (yield 56%) was obtained according to the procedure for compound 9 except with the use of 1,4-diiodobutane: [α]_{D}²⁰=−186.50 (c=0.25 in MeOH); ¹H NMR (400 MHz, CDCl₃): δ=6.92 (d, J=8.0 Hz, 1H), 6.62 (brs, 1H), 6.51 (d, J=2.0 Hz, 1H), 6.40 (dd, J=2.0, 8.0 Hz, 1H), 3.93 (t, J=8.0 Hz, 2H), 3.89 (brs, 1H), 3.70 (dd, J=4.0, 12.0 Hz, 1H), 3.54-3.49 (m, 2H), 3.05-3.01 (m, 1H), 2.95 (t, J=8.0 Hz, 2H), 2.79 (s, 3H), 2.79-2.76 (m, 1H), 2.48-2.41 (m, 1H), 2.34 (s, 3H), 1.86-1.74 (m, 4H), 1.05 (d, J=8.0 Hz, 3H), 0.87 ppm (d, J=8.0 Hz, 3H); ¹³C NMR (100 MHz, CDCl₃): δ=195.9, 173.7, 158.7, 152.6, 133.3, 123.0, 106.5, 106.3, 69.7, 67.2, 66.0, 54.5, 36.6, 34.9, 30.6, 28.8, 28.4, 28.2, 26.3, 20.5, 19.8 ppm; ESI-HRMS calculated for [C₂₁H₃₂N₂O₄S+H]⁺: 409.2161, found: 409.2164; HPLC purity: 95.1%.

Thioacetic acid S-[5-(5-hydroxymethyl-2-isopropyl-1-methyl-3-oxo-1,2,3,4,5,6-hexahydrobenzo[e][1,4]diazocin-9-yloxy)pentyl] ester (11): Compound 11 (yield 38%) was obtained according to the procedure for compound 9 except with the use of 1,5-diiodopentane: [α]_{D}²⁰=−261.70 (c=0.45 in MeOH); ¹H NMR (400 MHz, CDCl₃): δ=6.92 (d, J=8.0 Hz, 1H), 6.74 (brs, 1H), 6.51 (d, J=2.0 Hz, 1H), 6.40 (dd, J=2.0, 8.0 Hz, 1H), 3.91 (t, J=8.0 Hz, 2H), 3.89 (brs, 1H), 3.70 (dd, J=4.0, 8.0 Hz, 1H), 3.54-3.49 (m, 2H), 3.05-3.01 (m, 1H), 2.90 (t, J=8.0 Hz, 2H), 2.78 (s, 3H), 2.78-2.70 (m, 1H), 2.48-2.41 (m, 1H), 2.33 (s, 3H), 1.86-1.76 (m, 2H), 1.67-1.61 (m, 2H), 1.55-1.49 (m, 2H), 1.05 (d, J=8.0 Hz, 3H), 0.87 ppm (d, J=8.0 Hz, 3H); ¹³C NMR (100 MHz, CDCl₃): δ=195.9, 174.0, 158.7, 152.6, 132.3, 123.0, 106.6, 106.3, 69.8, 67.5, 65.8, 54.5, 36.5, 35.0, 30.6, 29.3, 28.9, 28.8, 28.2, 25.3, 20.5, 19.9 ppm; ESI-HRMS calculated for [C₂₂H₃₄N₂O₄S+H]⁺: 423.2318, found: 423.2317; HPLC purity: 99.5%.

Thioacetic acid S-[6-(5-hydroxymethyl-2-isopropyl-1-methyl-3-oxo-1,2,3,4,5,6-hexahydrobenzo[e][1,4]diazocin-9-yloxy)hexyl] ester (12): The compound 12 (yield 67%) was obtained according to the procedure for compound 9 except with the use of 1,6-diiodohexane: [α]_{D}²⁰=−197.20 (c=0.55 in MeOH); ¹H NMR (400 MHz, CDCl₃): δ=6.92 (d, J=8.4 Hz, 1H), 6.52 (d, J=2.0 Hz, 1H), 6.39 (dd, J=2.0, 8.0 Hz, 1H), 6.20 (brs, 1H), 3.92-3.89 (m, 3H), 3.76-3.70 (m, 1H), 3.52-3.50 (m, 2H), 3.06 (dd, J=6.8, 16.4 Hz, 1H), 2.88 (t, J=7.2 Hz, 2H), 2.80 (s, 3H), 2.80-2.70 (m, 1H), 2.45-2.43 (m, 1H), 2.33 (s, 3H), 1.78-1.74 (m, 2H), 1.62-1.56 (m, 2H), 1.47-1.43 (m, 4H), 1.05 (d, J=6.4 Hz, 3H), 0.87 ppm (d, J=6.4 Hz, 3H); ¹³C NMR (100 MHz, CDCl₃): δ=196.1, 174.1, 158.8, 152.6, 132.3, 122.9, 106.7, 106.4, 69.9, 67.8, 65.9, 54.6, 36.6, 35.1, 30.7, 29.5, 29.2, 29.0, 28.6, 28.3, 25.6, 20.5, 19.9 ppm; ESI-HRMS calculated for [C₂₃H₃₆N₂O₄S+H]⁺: 437.2474, found: 437.2478; HPLC purity: 99.9%.

Thioacetic acid S-[7-(5-hydroxymethyl-2-isopropyl-1-methyl-3-oxo-1,2,3,4,5,6-hexahydrobenzo[e][1,4]diazocin-9-yloxy)heptyl] ester (13): Compound 13 (yield 49%) was obtained according to the procedure for compound 9 except with the use of 1,7-ditosylheptane: [α]_{D}²⁰=−183.43 (c=0.18 in MeOH); ¹H NMR (400 MHz, CDCl₃): δ=6.93 (d, J=8.0 Hz, 1H), 6.88 (brs, 1H), 6.54 (d, J=2.0 Hz, 1H), 6.43 (dd, J=2.0, 8.0 Hz, 1H), 4.01 (brs, 1H), 3.91 (t, J=8.0 Hz, 2H), 3.74 (dd, J=4.0 Hz, 1H), 3.59-3.51 (m, 2H), 3.03 (dd, J=8.0, 16.0 Hz, 1H), 2.87 (t, J=8.0 Hz, 2H), 2.79 (s, 3H), 2.79-2.76 (m, 1H), 2.45-2.43 (m, 1H), 2.32 (s, 3H), 1.78-1.72 (m, 2H), 1.60-1.57 (m, 2H), 1.47-1.38 (m, 6H), 1.06 (d, J=8.0 Hz, 3H), 0.89 ppm (d, J=8.0 Hz, 3H); ¹³C NMR (100 MHz, CDCl₃): δ=196.1, 174.3, 158.9, 152.6, 132.3, 122.9, 107.0, 106.6, 70.3, 67.9, 65.9, 54.6, 36.5, 36.3, 30.7, 29.4, 29.2, 29.1, 28.9, 28.7, 28.3, 25.9, 20.3, 19.9 ppm; ESI-HRMS calculated for [C₂₄H₃₈N₂O₄S+Na]⁺: 473.2450, found: 473.2443; HPLC purity: 98.5%.

Thioacetic acid S-[8-(5-hydroxymethyl-2-isopropyl-1-methyl-3-oxo-1,2,3,4,5,6-hexahydrobenzole[e][1,4]diazocin-9-yloxy)octyl] ester (14): Compound 14 (yield 39%) was obtained according to the procedure for compound 9 except with the use of 1,8-dibromooctane: [α]_{D}²⁰=−154.37 (c=0.07 in MeOH); ¹H NMR (400 MHz, CDCl₃): δ=6.94 (d, J=8.0 Hz, 1H), 6.93 (brs, 1H), 6.55 (d, J=2.4 Hz, 1H), 6.44 (dd, J=2.4, 8.0 Hz, 1H), 4.00 (brs, 1H), 3.92 (t, J=6.4 Hz, 2H), 3.73 (dd, J=4.0, 11.2 Hz, 1H), 3.58-3.51 (m, 2H), 3.03-2.99 (m, 1H), 2.88 (t, J=72 Hz, 2H), 2.80 (s, 3H), 2.80-2.76 (m, 1H), 2.48-2.41 (m, 1H), 2.34 (s, 3H), 1.79-1.75 (m, 2H), 1.60-1.57 (m, 2H), 1.46-1.36 (m, 8H), 1.07 (d, J=6.8 Hz, 3H), 0.90 ppm (d, J=6.8 Hz, 3H); ¹³C NMR (100 MHz, CDC₁₃): δ=196.1, 174.4, 158.9, 152.6, 132.3, 122.9, 106.9, 106.6, 70.1, 67.9, 65.8, 54.6, 36.5, 35.2, 30.6, 29.5, 29.3, 29.2, 29.1, 29.0, 28.7, 28.3, 26.0, 20.3, 19.9 ppm; ESI-HRMS calculated for [C₂₅H₄₀N₂O₄S+H]⁺: 465.2787, found: 465.2787; HPLC purity: 98.4%.

The effectiveness, or potency, of a benzolactam of structural formula (I) with respect to activating of PKC and inhibiting HDAC is measured using standard assays in the art, as set forth and discussed below.

sAPPα and Aβ₄₀ detection: HEK93 cell lines stably transfected with human APP were maintained in Dulbecco's modified Eagle's medium (DMEM) with 10% fetal bovine serum (FBS). Cells were seeded onto 6-well plates, and incubated at 37° C. overnight. Compounds 9-14 were first dissolved in dimethyl sulfoxide (DMSO) as a 10 mM stock and diluted into the respective concentrations with fresh DMEM with 10% FBS. After exposure overnight, the cell medium was replaced by fresh medium containing the PKC agonist. After incubation for 48 hours, the medium was collected, and both the soluble APP (sAPPα) and Aβ₄₀ levels in the medium were determined by using the sAPPα and Aβ₄₀ ELISA kits obtained from Biosource, Camarillo, Calif. according to the manufacturer's instructions.

Primary neurons and cell culture: Cell cultures were obtained from the cerebral cortex of fetal Sprague-Dawley rats (embryonic day 17), as described previously [31]. All experiments were initiated 24 hours after plating. Under these conditions, the cells are not susceptible to glutamate-mediated excitotoxicity.

Oxidative toxicity and neuron viability assays: For cytotoxicity studies, cells were rinsed with warm phosphate-buffered saline (PBS), then placed in minimum essential medium (Invitrogen, Carlsbad, Calif.) containing glucose (5.5 g L⁻¹), 10% fetal calf serum, L-glutamine (2 mM), and cysteine (100 μM). Oxidative stress was induced by the addition of the glutamate analogue HCA (5 M) to the media. HCA was diluted from 100-fold concentrated solutions that were adjusted to pH 7.5. In combination with HCA, TSA, SAHA, or the novel PKC-HDAC compounds were added at various concentrations. Viability was assessed after 48 hours by calceinacetoxymethyl ester (AM)/ethidium homodimer-1 staining (live/dead assay; Molecular Probes, Eugene, Oreg., USA) under fluorescence microscopy and the MTT assay method. Each bar represents the mean ±SEM of four replicates.

Histone precipitation and Western blot analysis: Treated neurons (about 1×10⁶) were incubated in hypotonic lysis buffer (1 mL) containing Tris-HCl (pH 8.0, 10 mM), KCl (1 mM), MgCl₂ (1.5 mm), DTT (1 mM), aprotinin (1 mM), pepstatin (1 mM), and PMSF (0.4 mM) for 30 minutes, rotating at 4° C. Nuclei were pelleted by centrifugation for 10 minutes at 10,000 rpm (9000 g), resuspended in H₂SO₄ (0.4N, 200 μL), and rotated for 12 hours at 4° C. Following centrifugation at 13,000 rpm (16,000 g value) for 10 minutes, the supernatant was transferred to anew tube, and the histone proteins were precipitated by adding 100% trichloroacetic acid (TCA: 66 μL) dropwise followed by 30 minutes incubation on ice. Histone proteins were pelleted by centrifugation at 13,000 rpm (16,000 g) for 10 minutes, washed twice with ice-cold acetone, dried at room temperature for 20-40 minutes, and resuspended in H₂O (50 mL). Total histone proteins (20 μL) were boiled in Laemmli buffer and electrophoresed under reducing conditions on 15% polyacrylamide gel. Histone proteins were transferred to a nitrocellulose membrane (Bio-Rad, Hercules, Calif.). Nonspecific binding was inhibited by incubation in Tris-buffered saline with Tween 20 (TBST: 50 mM Tris-HCl pH 8.0, 0.9% NaCl, and 0.1% Tween 20) containing 5% nonfat dried milk for at least 1.5 hours. Primary antibodies against acetylated histone H4 or total histone H4 (Upstate) were diluted 1:1000 or 1:4000, respectively, in TBST containing 5% milk and incubated with the membrane for 3 hours at room temperature, followed by incubation with anti-rabbit horseradish peroxidase-conjugated secondary antibodies for 2 h at room temperature. Acetyl histone H4 and total histone H4 immunoreactivity was detected according to the enhanced chemiluminescent protocol (Amersham Biosciences, Fairfield, Conn.).

In accordance with the present invention, the benzolactam compounds showing PKC activation and HDAC inhibition were tested to show that such compounds provide a novel method in the treatment of AD. The PKC-activating properties of the present compounds enhance the α-secretase pathway in the processing of amyloid precursor protein (APP), while their HDAC inhibiting properties confer neuroprotective activity. The present benzolactams caused a concentration-dependent increase in sAPPα and decrease in β-amyloid (Aβ) production in the concentration range of 0.1-10 μM, consistent with a shift of APP metabolism toward the a-secretase-processing pathway.

Moreover, the tested benzolactam compounds showed neuroprotective effects in the 10-20 μM range in the homocystcate (HCA) cortical neuron model of oxidative stress. It was found that the tested benzolactam compounds caused increased levels of histone acetylation (H4), thus indicating an ability to inhibit HDAC activity.

The compounds studied also showed nanomolar binding affinities for PKC demonstrating that the present benzolactam compounds combine both PKC-activating properties with HDAC inhibitory properties. Such agents therefore are capable of modulating amyloid processing while showing neuroprotection.

To demonstrate that the benzolactam compounds of the present invention retain α-secretase-activating properties, while also exhibiting a neuroprotective action through HDAC inhibition, various benzolactams were prepared. Benzolactams previously were shown to have high binding affinity for PKC and to function as an agonist, especially when lipophilic side chains capable of interacting with the cell membrane are present [26]. High structural diversity is possible for this side chain because it does not interact with the diacylglycerol (DAG) recognition site of PKC. Therefore, the moiety Y-Z of the present compounds are functional groups capable of interacting with the zinc cation present in the HDAC catalytic site.

It was found that some thiol-containing benzolactams are less toxic, and therefore are preferred over hydroxamate-coating benzolactams at protecting cortical neurons from oxidative stress [27]. Accordingly, thioacetate and lipoic acid groups are preferred zinc binding groups (ZBGs), i.e., Y-Z moieties.

ClogP and topological polar surface area (tPSA) values were calculated (Table 1). ClogP values for the tested compounds ranged from 0.92 to 4.67. The tPSA values were in the range of 61.80-90.90. These values, combined with in vivo studies of other benzolactam compounds, indicate good cellular permeability and blood-brain barrier penetration for the tested compounds, designed to possess PKC-activating and HDAC-inhibitory activity [17]. The binding affinities of the compounds for mouse PKCα were determined as described previously [26]. The Kᵢ values for each of the compounds 2, 3, 6, 7, and 9-14, and known HDAC inhibitors bryostatin 1 and SAHA are listed in Table 1.

TABLE 1
PKC binding affinity of the benzolactams together with their
calculated logP values and topological polar surface areas.

| Compd | ClogP^{[a]} | tPSA^{[b]} | K₁[nm] ± SEM |
|---|---|---|---|
| 2 | 0.92 | 72.79 | 4840 ± 320 |
| 3 | 4.65 | 61.80 | 5.4 ± 0.1 |
| 6 | 4.11 | 78.87 | 148 ± 18 |
| 7 | 4.66 | 90.90 | 5.7 ± 1.0 |
| 9 | 2.21 | 78.87 | 21.0 ± 2.8 |
| 10 | 2.70 | 78.87 | 15.8 ± 0.8 |
| 11 | 3.19 | 78.87 | 6.6 ± 0.2 |
| 12 | 3.68 | 78.87 | 8.7 ± 0.7 |
| 13 | 4.17 | 78.87 | 4.5 ± 0.4 |
| 14 | 4.67 | 78.87 | 2.8 ± 0.8 |
| Bryostatin 1 | 3.80 | 240.14 | 1.4 ± 0.2^{[52]} |
| SAHA | 1.44 | 78.42 | — |

^{[a]}ClogP values (KOWWIN) were calculated from http://146.107.217.178/lab/alogps/start.html (accessed Apr. 2, 2009).
^{[b]}tPSA values were calculated from http://www.molinspiration.com/cgi-bin/properties?textMode=1 (accessed Apr. 2, 2009).

It was found that the present benzolactam compounds possess PKC-activating and HDAC-inhibitory activities, increase sAPPα production, and decrease Aβ production.

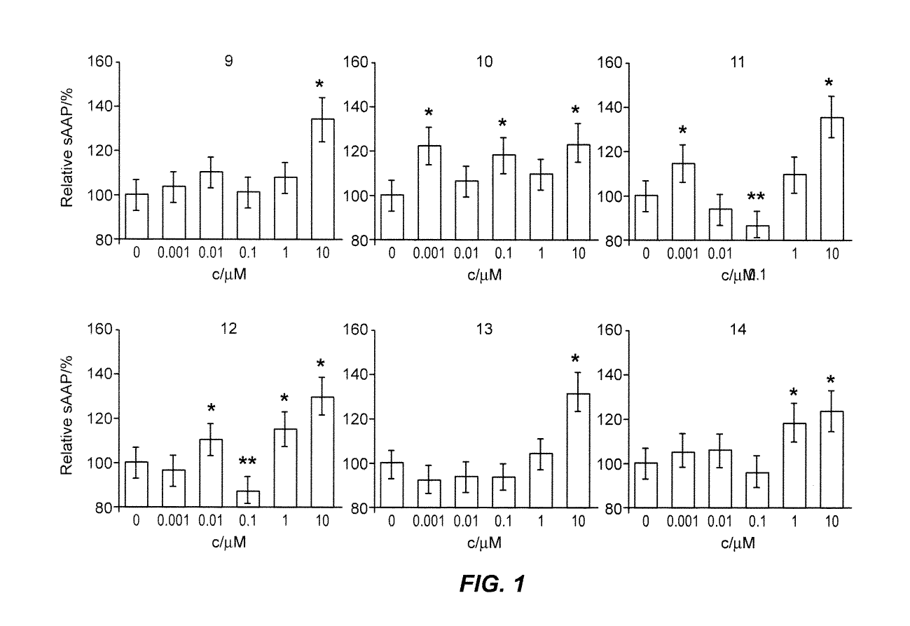

Activation of PKC in fibroblasts has been shown to increase α-secretase-mediated cleavage of the amyloid precursor protein, and thus increase production of the non-amyloidogenic sAPPα [17]. To demonstrate the effect of a present benzolactam on altering sAPPα production and release, HEK293 cells stably transfected with human APP were treated with compounds 9-14 at concentrations ranging from 0.001 to 10 μM, and sAPPα levels were measured by sAPPα ELISA. Treatment with compounds 12 and 14 resulted in an increase in sAPPα levels at concentrations of 1-10 μm (FIG. 1). In FIG. 1, the data are presented as percent of control±SEM. The “*” signifies a significantly higher percent than control without compound (p<0.01), and “**” signifies a significantly lower percent than control without a compound (p<0.01). Treatment with compounds 9, 11, and 13 resulted in an increase in sAPPα levels at a concentration of 10 μm. In contrast, compound 10 effected a statistically significant elevation in sAPPα at a concentration of 1 nM.

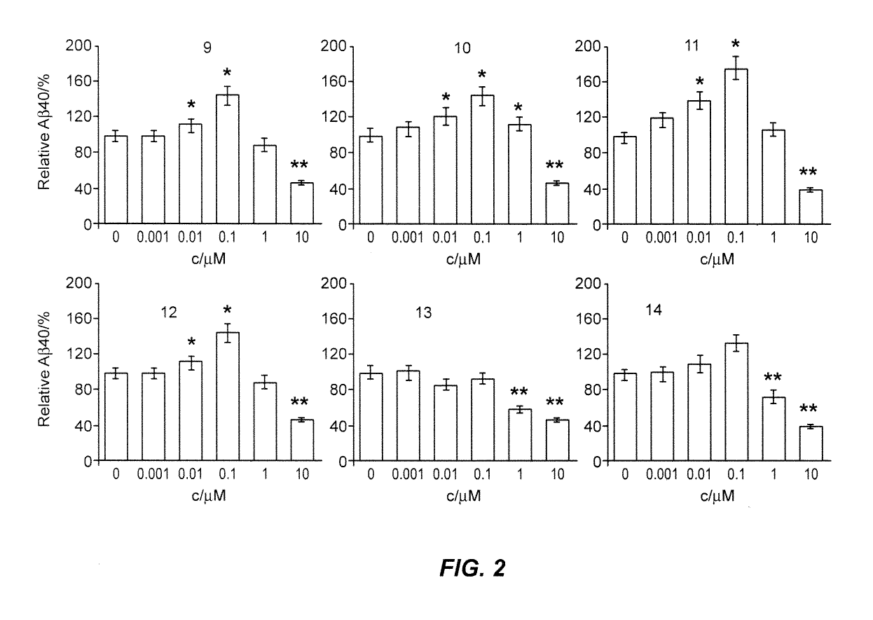

It is theorized, but not relied upon, that if APP synthesis is held relatively constant, the production of Aβ decreases if more of the APP is processed through the α-secretase pathway. To confirm that treatment with a present benzolactam enhances the α-secretase APP-processing pathway, compounds 9-14 were examined to determine whether the increased levels of sAPPα corresponds to a decrease in the production of Aβ₄₀. The cells were treated for 24 h at concentrations ranging from 0.001 to 10 μM, and Aβ₄₀ levels were measured by Aβ₄₀ ELISA. Treatment with compounds 13 and 14 resulted in a significant decrease in Aβ₄₀ levels at concentrations of 1-10 μM (FIG. 2). In FIG. 2, the data are presented as in FIG. 1, with “*” and “**” having the same meaning. Treatment with compounds 9, 10, 11, and 12 resulted in a significant decrease in Aβ₄₀ levels at 10 μM. In general, there was good agreement between an ability of a present benzolactam to increase sAPPα and its ability to decrease Aβ₄₀.

The present benzolactams compounds, designed to exhibit PKC activation and HDAC inhibition, induce histone H4 acetylation and protect cortical neurons from oxidative-stress-induced death.

Oxidative stress has been implicated to play a crucial role in the pathogenesis of AD and has been observed in the brains of AD patients. Indeed, oxidative stress occurs early in the progression of Alzheimer's disease, even before the development of the pathologic hallmarks, neurofibrillary tangles, and senile plaques. It has been demonstrated that HDAC inhibition can protect against neuronal oxidative stress in vitro.

The dynamic equilibria present between the acetylated and deacetylated states of lysine residues of histone proteins are regulated by HAT and HDAC activity. By blocking deacetylation through the application of HDAC inhibitors, the global acetylation level of the histones in neurons can be increased, and profound changes in gene expression can occur. Certain thiol-based HDAC inhibitors are less toxic, and therefore are preferred over hydroxamate-based counterparts at protecting cortical neurons from oxidative stress [27].

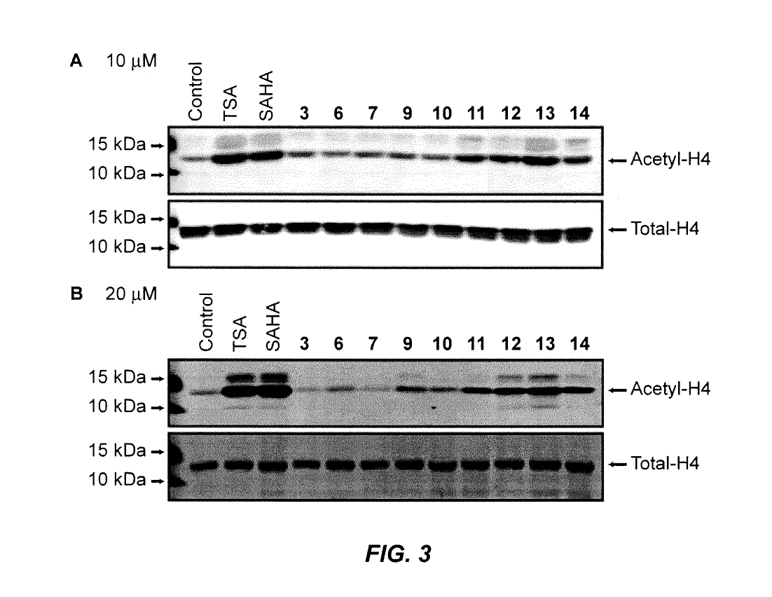

To illustrate the HDAC-inhibitory activity of the present benzolactams, an ability of the compounds to alter histone acetylation levels was examined. FIG. 3 contains Western blot analyses showing the effect of percent benzolactam compounds at 10 μM (FIG. 3A) and 20 μM (FIG. 3B) in the acetylation levels of histone H4. Cultures of primary cortical neurons were treated with the various benzolactam compounds (at 10 μM) for a period of 8 hours, and Western blot analysis then was performed using acetyl-histone H4-specific antibodies (FIG. 3A).

Compounds 11-14 each induced an increase in histone H4 acetylation at this concentration, consistent with inhibiting HDAC activity. For compounds 12 and 13, histone H4 acetylation was similar to that induced by the prototypical HDAC inhibitors trichostatin A (TSA) and suberoylanilide hydroxamic acid (SAHA). Compounds 9 and 10 were less effective at inducing histone H4 acetylation at 10 μM, but modest acetylation was observed at 20 μM (FIG. 3B).

As expected, treatment of primary cortical neurons with compound 3 did not increase histone H4 acetylation, even at 20 μM, despite this compound's high affinity for PKC. This further illustrates the effect of the sulfur-bearing zinc binding groups for HDAC inhibition. Compounds 6 and 7, which contain a lipoic acid side chain, also failed to induce histone H4 acetylation in these cells. While lipoic acid has been shown to act as an HDAC inhibitor [29], this requires initial reduction to the dithiol, which may be more difficult in this class of compounds. Therefore, the observed degree of histone H4 acetylation may correlate with the ability of the free thiol, derived from an acetylthio prodrug form by the action of esterases, to engage the catalytic zinc ion present at the bottom of the gorge region of the HDAC enzyme [30].

Having showed that compounds 9-14 display HDAC-inhibitory activity, their ability to protect cultured primary cortical neurons from oxidative stress-induced death was investigated. In this model of neurodegeneration, oxidative stress is induced by the presence of the glutamate analogue homocysteate (HCA) at a concentration of 5 mM, which depletes the cellular antioxidant glutathione through competitive inhibition of cysteine uptake at the level of the plasma membrane cysteine/glutamate antiporter system xc-. Because cysteine is required for the synthesis of glutathione, the inhibition of its uptake results in glutathione depletion. Cellular redox homeostasis, therefore, becomes disrupted with the accumulation of endogenously produced and unopposed oxidants, resulting in neuronal degeneration over a period of about 24 hours. Importantly, this model uses embryonic primary neurons (E17), which, at this early developmental stage, lack ionotropic and metabotropic receptors and are not susceptible to excitotoxicity. Instead, death is induced by the accumulation of unopposed free radicals, and the neurons exhibit a number of apoptotic features [31].

Primary cortical neurons were treated with compounds 3, 6, 7, and 9-14 at concentrations ranging from 1 to 20 μM in the presence or absence of HCA for 48 hours, well beyond the time for degeneration to occur in this model. As shown in FIG. 3, in the absence of HCA, no tested compounds showed any toxicity in neuron viability measurements by MTT assay, even at the highest concentrations tested (20 μM). For comparison, the effect of the commercially available prototypical HDAC inhibitor, TSA again was carried out in the same studies. In contrast to the test compounds and as previously published [32], TSA showed considerable dose dependent toxicity in the absence of HCA. In the presence of HCA, compounds 9-14, each with a sulfur atom in the side chain, showed 100% neuroprotection from HCA-induced oxidative toxicity at 20 μM, correlating with the ability of these compounds to induce histone acetylation. Indeed, within this set, compounds 11-13 showed complete protection at 10 μM, corresponding to their ability to induce histone H4 acetylation at the same concentration.

In contrast to the protection observed with compounds 9-14, compounds 3, 6, and 7 showed no neuroprotection up to at 20 μM. In the case of compound 3, which has high affinity for PKC, this again illustrates the effect of the sulfur-bearing zinc binding groups for HDAC inhibition, and the capacity for HDAC inhibition to protect neurons from oxidative stress-induced death. Indeed, the lack of protection in this system with the lipoic acid side chain containing compounds 6 and 7, which also failed to induce histone H4 acetylation, further illustrate the effect of a sulfur absorbing zinc binding group.

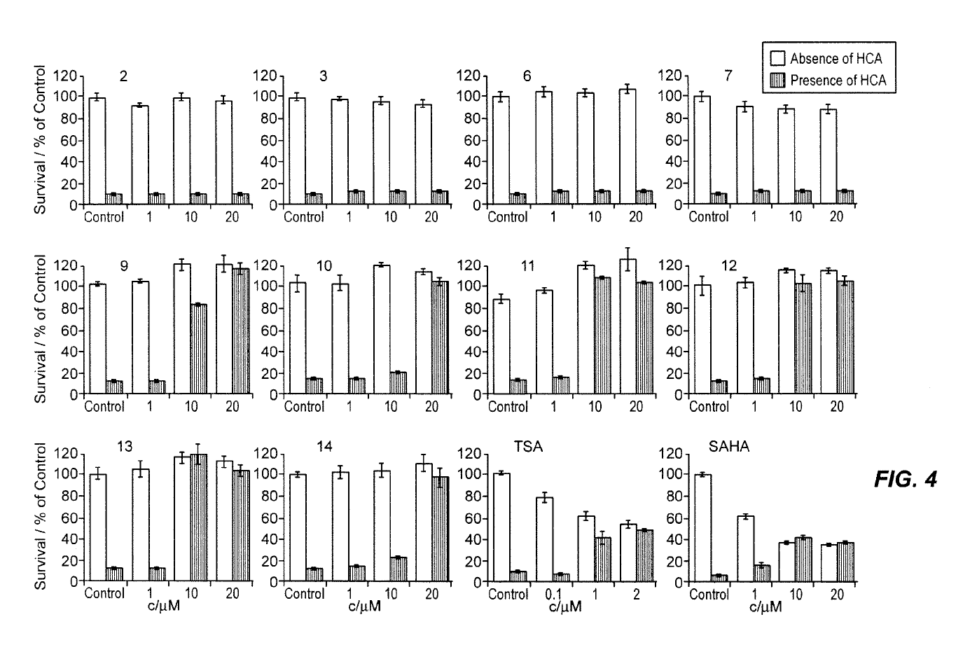

In one embodiment, the present invention relates to a method of treating an individual suffering from a disease or condition wherein activation of PKC and inhibition of HDAC provides a benefit comprising administering a therapeutically effective amount of a compound of structural formula (I) to an individual in need thereof. See FIG. 4 showing the dose dependent survival of cortical neurons upon exposure to present benzolactam compounds, TSA, and SAHA in the absence or presence should of HCA. Data in FIG. 4 are presented as percent of control±SEM. Neuron survival was quantified by MTT assay, a colorimetric assay in which the amount of yellow MTT is reduced to purple formazan by active mitochondrial reductase enzymes in viable cells.

The methods described herein relate to the use of a benzolactam of structural formula (I) and an optional second therapeutic agent useful in the treatment of diseases and conditions wherein activation of PKC and inhibition of HDAC provides a benefit. The methods of the present invention can be accomplished by administering a benzolactam of structural formula (I) as the neat compound or as a pharmaceutical composition. Administration of a pharmaceutical composition, or neat benzolactam of structural formula (I), can be performed during or after the onset of the disease or condition of interest. Typically, the pharmaceutical compositions are sterile, and contain no toxic, carcinogenic, or mutagenic compounds that would cause an adverse reaction when administered.

In many embodiments, a benzolactam of structural formula (I) is administered in conjunction with a second therapeutic agent useful in the treatment of a disease or condition wherein activation of PKC and inhibition of HDAC provides a benefit. The second therapeutic agent is different from the benzolactam of structural formula (I). A benzolactam of structural formula (I) and the second therapeutic agent can be administered simultaneously or sequentially. In addition, a benzolactam of structural formula (I) and second therapeutic agent can be administered from a single composition or two separate compositions. A benzolactam of structural formula (I) and the second therapeutic agent can be administered simultaneously or sequentially to achieve the desired effect.

The second therapeutic agent is administered in an amount to provide its desired therapeutic effect. The effective dosage range for each second therapeutic agent is known in the art, and the second therapeutic agent is administered to an individual in need thereof within such established ranges.

For example, in the treatment of Alzheimer's Disease, the second therapeutic agent can be, for example, memantine (NAMENDA®), galantamine (RAZADYNE®), rivastigmine (EXELON®), donepezil (ARICEPT®), tacrine (COGNEX®), or mixtures thereof.

The present invention therefore is directed to compositions and methods of treating diseases or conditions wherein activation of PKC and inhibition of HDAC provides a benefit. The present invention also is directed to pharmaceutical compositions comprising a benzolactam of structural formula (I) and a second therapeutic agent useful in the treatment of diseases and conditions wherein activation of PKC and inhibition of HDAC provides a benefit. Further provided are kits comprising a benzolactam of structural formula (I) and, optionally, a second therapeutic agent useful in the treatment of diseases and conditions wherein activation of PKC and inhibition of HDAC provides a benefit, packaged separately or together, and an insert having instructions for using these active agents.

A benzolactam of structural formula (I) and the second therapeutic agent can be administered together as a single-unit dose or separately as multi-unit doses, wherein a benzolactam of structural formula (I) is administered before the second therapeutic agent or vice versa. One or more dose of a benzolactam of structural formula (I) and/or one or more dose of the second therapeutic agent can be administered. A benzolactam of structural formula (I) therefore can be used in conjunction with one or more second therapeutic agents.

The benzolactams of structural formula (I) therefore are useful for treating a neurological disease by administration of amounts of a benzolactam of structural formula (I) effective to treat the neurological disease or by administration of a pharmaceutical composition comprising amounts of a benzolactam of structural formula (I) effective to treat the neurological disease. The neurological diseases that can be treated include, but are not limited to, Huntington's disease, lupus, schizophrenia, multiple sclerosis, muscular dystrophy, dentatorubralpallidoluysian atrophy (DRRLA), spinal and bulbar muscular atrophy (SBMA), fine spinocerebellar ataxias (SCA1, SCA2, SCA3/MJD (Machado-Joseph Disease), SCA6, and SCA7), drug-induced movement disorders, Creutzfeldt-Jakob disease, amyotrophic lateral sclerosis, Pick's disease, Alzheimer's disease, Lewy body dementia, cortico basal degeneration, dystonia, myoclonus, Tourette's syndrome, tremor, chorea, restless leg syndrome, Parkinson's disease, Parkinsonian syndromes, anxiety, depression, psychosis, manic depression, Friedreich's ataxia, Fragile X syndrome, spinal muscular dystrophy, Rett syndrome, Rubinstein-Taybi syndrome, Wilson's disease, and multi-infarct state.

In a preferred embodiment, the neurological disease treated is Huntington's disease, Parkinson's disease, Alzheimer's disease, spinal muscular atrophy, lupus, or schizophrenia. In a most preferred embodiment, the neurological disease treated is Alzheimer's disease.

In the present method, a therapeutically effective amount of one or more benzolactam of structural formula (I), typically formulated in accordance with pharmaceutical practice, is administered to a human being in need thereof. Whether such a treatment is indicated depends on the individual case and is subject to medical assessment (diagnosis) that takes into consideration signs, symptoms, and/or malfunctions that are present, the risks of developing particular signs, symptoms and/or malfunctions, and other factors.

A benzolactam of structural formula (I) can be administered by any suitable route, for example by oral, buccal, inhalation, topical, sublingual, rectal, vaginal, intracisternal or intrathecal through lumbar puncture, transurethral, nasal, percutaneous, i.e., transdermal, or parenteral (including intravenous, intramuscular, subcutaneous, intracoronary, intrademial, intramammary, intraperitonea), intraarticular, intrathecal, retrobulbar, intrapulmonary injection and/or surgical implantation at a particular site) administration. Parenteral administration can be accomplished using a needle and syringe or using a high pressure technique.

Pharmaceutical compositions include those wherein a benzolactam of structural formula (I) is present in a sufficient amount to be administered in an effective amount to achieve its intended purpose. The exact formulation, route of administration, and dosage is determined by an individual physician in view of the diagnosed condition or disease. Dosage amount and interval can be adjusted individually to provide levels of a benzolactam of structural formula (I) that is sufficient to maintain therapeutic effects.

Toxicity and therapeutic efficacy of the compounds of structural formula (I) can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index, which is expressed as the ratio between LD₅₀ and ED₅₀. Compounds that exhibit high therapeutic indices are preferred. The data obtained from such data can be used in formulating a dosage range for use in humans. The dosage preferably lies within a range of circulating compound concentrations that include the ED₅₀ with little or no toxicity. The dosage can vary within this range depending upon the dosage form employed, and the route of administration utilized. Determination of a therapeutically effective amount is well within the capability of those skilled in the art, especially in light of the detailed disclosure provided herein.

A therapeutically effective amount of a benzolactam of structural formula (I) required for use in therapy varies with the nature of the condition being treated, the length of time that activity is desired, and the age and the condition of the patient, and ultimately is determined by the attendant physician. Dosage amounts and intervals can be adjusted individually to provide plasma levels of the benzolactam that are sufficient to maintain the desired therapeutic effects. The desired dose conveniently can be administered in a single dose, or as multiple doses administered at appropriate intervals, for example as one, two, three, four or more subdoses per day. Multiple doses often are desired, or required. For example, a present benzolactam can be administered at a frequency of: four doses delivered as one dose per day at four-day intervals (q4d×4); four doses delivered as one dose per day at three-day intervals (q3d×4); one dose delivered per day at five-day intervals (qd×5); one dose per week for three weeks (qwk3); five daily doses, with two days rest, and another five daily doses (5/2/5); or, any dose regimen determined to be appropriate for the circumstance.

The dosage of a composition containing a benzolactam of structural formula (I), or a composition containing the same, can be from about 1 ng/kg to about 200 mg/kg, about 1 μg/kg to about 100 mg/kg, or about 1 mg/kg to about 50 mg/kg of body weight. The dosage of a composition may be at any dosage including, but not limited to, about 1 μg/kg, 10 μg/kg, 25 μg/kg, 50 μg/kg, 75 μg/kg, 100 μg/kg, 125 μg/kg, 150 μg/kg, 175 μg/kg, 200 μg/kg, 225 μg/kg, 250 μg/kg, 275 μg/kg, 300 μg/kg, 325 μg/kg, 350 μg/kg, 375 μg/kg, 400 μg/kg, 425 μg/kg, 450 μg/kg, 475 μg/kg, 500 μg/kg, 525 μg/kg, 550 μg/kg, 575 μg/kg, 600 μg/kg, 625 μg/kg, 650 μg/kg, 675 μg/kg, 700 μg/kg, 725 μg/kg, 750 μg/kg, 775 μg/kg, 800 μg/kg, 825 μg/kg, 850 μg/kg, 875 μg/kg, 900 μg/kg, 925 μg/kg, 950 μg/kg, 975 μg/kg, 1 mg/kg, 5 mg/kg, 10 mg/kg, 15 mg/kg, 20 mg/kg, 25 mg/kg, 30 mg/kg, 35 mg/kg, 40 mg/kg, 45 mg/kg, 50 mg/kg, 60 mg/kg, 70 mg/kg, 80 mg/kg, 90 mg/kg, 100 mg/kg, 125 mg/kg, 150 mg/kg, 175 mg/kg, or 200 mg/kg. The above dosages are exemplary of the average case, but there can be individual instances in which higher or lower dosages are merited, and such are within the scope of this invention. In practice, the physician determines the actual dosing regimen that is most suitable for an individual patient, which can vary with the age, weight, and response of the particular patient.

A benzolactam of structural formula (I) used in a method of the present invention typically is administered in an amount of about 0.005 to about 500 milligrams per dose, about 0.05 to about 250 milligrams per dose, or about 0.5 to about 100 milligrams per dose. For example, an HDACI of structural formula (I) can be administered, per dose, in an amount of about 0.005, 0.05, 0.5, 5, 10, 20, 30, 40, 50, 100, 150, 200, 250, 300, 350, 400, 450, or 500 milligrams, including all doses between 0.005 and 500 milligrams.

The benzolactams of the present invention typically are administered in admixture with a pharmaceutical carrier selected with regard to the intended route of administration and standard pharmaceutical practice. Pharmaceutical compositions for use in accordance with the present invention are formulated in a conventional manner using one or more physiologically acceptable carriers comprising excipients and auxiliaries that facilitate processing of HDACIs of structural formula (I).

The term “carrier” refers to a diluent, adjuvant, or excipient, with which a benzolactam of structural formula (I) is administered. Such pharmaceutical carriers can be liquids, such as water and oils, including those of petroleum, animal, vegetable or synthetic origin, such as peanut oil, soybean oil, mineral oil, sesame oil, and the like. The carriers can be saline, gum acacia, gelatin, starch paste, talc, keratin, colloidal silica, urea, and the like. In addition, auxiliary, stabilizing, thickening, lubricating and coloring agents can be used. The pharmaceutically acceptable carriers are sterile. Water is a preferred carrier when the benzolactam of structural formula (I) is administered intravenously. Saline solutions and aqueous dextrose and glycerol solutions can also be employed as liquid carriers, particularly for injectable solutions. Suitable pharmaceutical carriers also include excipients such as starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol, and the like. The present compositions, if desired, can also contain minor amounts of wetting or emulsifying agents, or pH buffering agents.

These pharmaceutical compositions can be manufactured, for example, by conventional mixing, dissolving, granulating, dragee-making, emulsifying, encapsulating, entrapping, or lyophilizing processes. Proper formulation is dependent upon the route of administration chosen. When a therapeutically effective amount of a benzolactam of structural formula (I) is administered orally, the composition typically is in the form of a tablet, capsule, powder, solution, or elixir. When administered in tablet form, the composition additionally can contain a solid carrier, such as a gelatin or an adjuvant. The tablet, capsule, and powder contain about 0.01% to about 95%, and preferably from about 1% to about 50%, of a benzolactam of structural formula (I). When administered in liquid form, a liquid carrier, such as water, petroleum, or oils of animal or plant origin, can be added. The liquid form of the composition can further contain physiological saline solution, dextrose or other saccharide solutions, or glycols. When administered in liquid form, the composition contains about 0.1% to about 90%, and preferably about 1% to about 50%, by weight, of a compound of structural formula (I).

When a therapeutically effective amount of a benzolactam of structural foil iula (I) is administered by intravenous, cutaneous, or subcutaneous injection, the composition is in the form of a pyrogen-free, parenterally acceptable aqueous solution. The preparation of such parenterally acceptable solutions, having due regard to pH, isotonicity, stability, and the like, is within the skill in the art. A preferred composition for intravenous, cutaneous, or subcutaneous injection typically contains, an isotonic vehicle. A benzolactam of structural formula (I) can be infused with other fluids over a 10-30 minute span or over several hours.

Benzolactams of structural formula (I) can be readily combined with pharmaceutically acceptable carriers well-known in the art. Such carriers enable the active agents to be formulated as tablets, pills, dragees, capsules, liquids, gels, syrups, slurries, suspensions and the like, for oral ingestion by a patient to be treated. Pharmaceutical preparations for oral use can be obtained by adding the benzolactam of structural formula (I) to a solid excipient, optionally grinding the resulting mixture, and processing the mixture of granules, after adding suitable auxiliaries, if desired, to obtain tablets or dragee cores. Suitable excipients include, for example, fillers and cellulose preparations. If desired, disintegrating agents can be added.

A benzolactam of structural formula (I) can be formulated for parenteral administration by injection, e.g., by bolus injection or continuous infusion. Formulations for injection can be presented in unit dosage form, e.g., in ampules or in multidose containers, with an added preservative. The compositions can take such forms as suspensions, solutions, or emulsions in oily or aqueous vehicles, and can contain formulatory agents such as suspending, stabilizing, and/or dispersing agents.

Pharmaceutical compositions for parenteral administration include aqueous solutions of the active agent in water-soluble form. Additionally, suspensions of a benzolactam of structural formula (I) can be prepared as appropriate oily injection suspensions. Suitable lipophilic solvents or vehicles include fatty oils or synthetic fatty acid esters. Aqueous injection suspensions can contain substances which increase the viscosity of the suspension. Optionally, the suspension also can contain suitable stabilizers or agents that increase the solubility of the compounds and allow for the preparation of highly concentrated solutions. Alternatively, a present composition can be in powder form for constitution with a suitable vehicle, e.g., sterile pyrogen-free water, before use.

A benzolactam of structural formula (I) also can be formulated in rectal compositions, such as suppositories or retention enemas, e.g., containing conventional suppository bases. In addition to the formulations described previously, the benzolactam of structural formula (I) also can be formulated as a depot preparation. Such long-acting formulations can be administered by implantation (for example, subcutaneously or intramuscularly) or by intramuscular injection. Thus, for example, the benzolactams of structural formula (I) can be formulated with suitable polymeric or hydrophobic materials (for example, as an emulsion in an acceptable oil) or ion exchange resins.

In particular, the benzolactams of structural formula (I) can be administered orally, buccally, or sublingually in the form of tablets containing excipients, such as starch or lactose, or in capsules or ovules, either alone or in admixture with excipients, or in the fowl of elixirs or suspensions containing flavoring or coloring agents. Such liquid preparations can be prepared with pharmaceutically acceptable additives, such as suspending agents. The benzolactams of structural formula (I) also can be injected parenterally, for example, intravenously, intramuscularly, subcutaneously, or intracoronarily. For parenteral administration, the benzolactams are best used in the form of a sterile aqueous solution which can contain other substances, for example, salts or monosaccharides, such as mannitol or glucose, to make the solution isotonic with blood.

As an additional embodiment, the present invention includes kits which comprise one or more compounds or compositions packaged in a manner that facilitates their use to practice methods of the invention. In one simple embodiment, the kit includes a compound or composition described herein as useful for practice of a method (e.g., a composition comprising a benzolactam of structural formula (I) and an optional second therapeutic agent), packaged in a container, such as a sealed bottle or vessel, with a label affixed to the container or included in the kit that describes use of the compound or composition to practice the method of the invention. Preferably, the compound or composition is packaged in a unit dosage form. The kit further can include a device suitable for administering the composition according to the intended route of administration, for example, a syringe, drip bag, or patch. In another embodiment, the compounds of structural formula (I) is a lyophilate. In this instance, the kit can further comprise an additional container which contains a solution useful for the reconstruction of the lyophilate.

### REFERENCES

1. A. Pascale, M. Amadio, S. Govoni, F. Battaini, Pharmacol. Res. 2007, 55, 560.
2. S. Gandy, P. Greengard, Int. Rev. Neurobiol, 1994, 36, 29.
3. J. D. Buxbaum, G. Thinakaran, V. Koliatsos, J. O'Callahan, H. H. Slunt, D. L. Price, S. S. Sisodia, J. Neurosci, 1998, 18, 9629.
4. J. D. Buxbaum, K. N. Liu, Y. Luo, J. L. Slack, K. L. Stocking, J. J. Peschon, R. S. Johnson, B. J. Castner, D. P. Cerretti, R. A. Black, J. Biol. Chem. 1998, 273, 27765.
5. G. Cole, K. R. Dobkins, L. A. Hansen, R. D. Terry, T. Saitoh, Brain Res. 1988, 452, 165.
6. E. Masliah, G. Cole, S. Shimohama, L. Hansen, R. DeTeresa, R. D. Terry, T. Saitoh, J. Neurosci. 1990, 10, 2113.
7. S. Shimohama, M. Narita, H. Matsushima, J. Kimura, M. Kameyama, M. Hagiwara, H. Hidaka, T. Taniguchi, Neurology 1993, 43, 1407.
8. H. Y. Wang, M. R. Pisano, E. Friedman, Neurobiol. Aging 1994, 15, 293.
9. E. Masliah, G. M. Cole, L. A. Hansen, M. Mallory, T. Albright, R. D. Terry, T. Saitoh, J. Neurosci. 1991, 11, 2759.
10. M. Chachin, S. Shimohama, Y. U. Kunugi, T. Taniguchi, Jpn. J. Pharmacol. 1996, 71, 175.
11. S. Govoni, S. Bergamaschi, M. Racchi, F. Battaini, G. Binetti, A. Bianchetti, M. Trabucchi, Neurology 1993, 43, 2581.
12. S. Govoni, M. Racchi, S. Bergamaschi, M. Trabucchi, F. Battaini, A. Bianchetti, G. Binetti, Ann. N. Y. Acad. Sci, 1996, 777, 332.
13. H. Cai, Y. Wang, D. McCarthy, H. Wen, D. R. Borchelt, D. L. Price, P. C. Wong, Nat. Neurosci. 2001, 4, 233.
14. R. Vassar, M. Citron, Neuron 2000, 27, 419.
15. S. Bhagavan, D. Ibarreta, D. Ma, A. P. Kozikowski, R. Etcheberrigaray, Neurobiol. Dis. 1998, 5,177.
16. D. Ibarreta, M. Duch3en, D. Ma, L. Qiao, A. P. Kozikowski, R. Etcheberrigaray, Neuroreport 1999, 10, 1035.
17. R. Etcheberrigaray, M. Tan, I. Dewachter, C. Kuiperi, I. Van der Auwera, S. Wera, L. Qiao, B. Bank, T. J. Nelson, A. P. Kozikowski, F. Van Leuven, D. L. Alkon, Proc. Natl. Acad. Sci. USA 2004, 101, 11141.
18. C. Y. Gui, L. Ngo, W. S. Xu, V. M. Richon, P. A. Marks, Proc. Natl. Acad. Sci. USA 2004, 101, 1241.
19. P. A. Marks, M. Dokmanovic, Expert Opin. Invest. Drugs 2005, 14, 1497.
20. S. Minucci, P. G. Pelicci, Nat. Rev. Cancer 2006, 6, 38.
21. S. G. Gray, F. Dangond, Epigenetics 2006, 1, 67.
22. T. Abel, R. S. Zukin, Curr. Opin. Pharmacol. 2008, 8, 57.
23. M. Minamiyama, M. Katsuno, H. Adachi, M. Waza, C. Sang, Y. Kobayashi, F. Tanaka, M. Doyu, A. Inukai, G. Sobue, Hum. Mol. Genet. 2004, 13, 1183.
24. A. P. Kozikowski, D. Ma, Y. P. Pang, P. Shum, V. Likic, P. K. Mishra, S. Macura, A. Basu, J. S. Lazo, R. G. Ball, J. Am. Chem. Soc. 1993, 115, 3957.
25. A. P. Kozikowski, S. Wang, D. Ma, J. Yao, S. Ahmad, R. I. Glazer, K. Bogi, P. Acs, S. Modarres, N. E. Lewin, P. M. Blumberg, J. Med. Chem. 1997, 40, 1316.
26. A. P. Kozikowski, 1. Nowak, P. A. Petukhov, R. Etcheberrigaray, A. Mohamed, M. Tan, N. Lewin, H. Hennings, L. L. Pearce, P. M. Blumberg, J. Med. Chem. 2003, 46, 364.
27. A. P. Kozikowski, Y. Chen, A. Gaysin, B. Chen, M. A. D'Annibale, C. M. Suto, B. C. Langley, J. Med. Chem. 2007, 50, 3054.
28, D. E. Clark, J. Pharm. Sci. 1999, 88, 815.
29. R. H. Dashwood, E. Ho, Semin. Cancer Biol, 2007, 17, 363.
30. T. A. Miller, D. J. Witter, S. Belvedere, J. Med. Chem. 2003, 46, 5097.
31. R. R. Ratan, T. H. Murphy, J. M. Baraban, J. Neurochem. 1994, 62, 376.B. Langley, M. A. D'Annibale, K. Suh, I. Ayoub, A. Tolhurst, B. Bastan, L. Yang, B. Ko, M. Fisher, S. Cho, M. F. Beal, R. R. Ratan, J. Neurosci. 2008, 28,163.
32. A. C. Newton, J. Biol. Chem. 1995, 270, 28495.

What is claimed:

1. A compound having a structural formula (I) 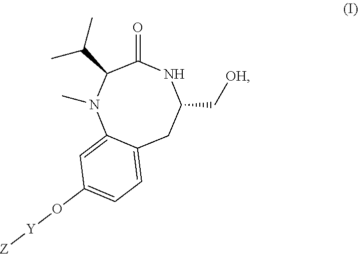
wherein Y is selected from the group consisting of null, C₁₋₈alkylene, NR^{b}, C(═O), C(═O)C₁₋₆alkylene, C₁₋₈alkyleneNR^{b}, C₁₋₄alkylenearyleneC₁₋₄alkylene, C₂₋₆alkenylene, C₄₋₈alkdienylene, C₁₋₆alkylenearylene, C(═O) arylene, C(═O)C₁₋₆alkylenearylene, C₁₋₆alkylenepiperidinyl, and C₂₋₆alkenylenearyleneC₁₋₄alkylene; and
Z is selected from the group consisting of 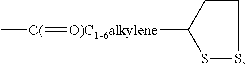
—C₃₋₁₀alkyleneSAc, —C(═O)N(R^{c})OH, 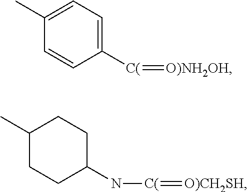
—SO₂NHR^{c}, —NHSO₂NHR^{c}, —NHSO₂C₁₋₆alkyl, —SO₂C₁₋₆alkyl, 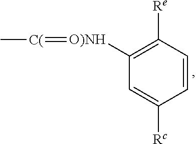
—C(═O)R^{f} wherein R^{f} is selected from the group consisting of OH, N(R^{c})², NH(OCH₃), N(CH₃)OH, C₁₋₆alkyl, CF₃, aryl, heteroaryl, C₃₋₈cycloalkyl, heterocycloalkyl, NHSO₂CH₃, NHSO₂CF₃, and C₁₋₆haloalkyl, —C(═O)(C(R^{c})²)₁₋₃SH, —SR^{d} wherein R^{d} is hydrogen or (C═O)CH₃, 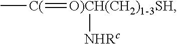
—S—(C═O)C₁₋₆alkyl, heterocycloalkyl optionally substituted with oxo (═O), thioxo (═S), or both, heteroaryl optionally substituted with —NH₂, —SH, or both, —N(H)C(═O)SH, —NHC(═O)NHR^{e}, —NHC(═O)CH₂R^{e}, —NHC(═O)(CH₂)₁₋₆SH, —NHC(═O)CH₂Hal, —NHC(═S)NHR^{e}, —NHC(═S)CH₂R^{e}, —C(═S)NHR^{e}, —C(═S)CH₂R^{e}, —NHC(═S)CH₂R^{e}, —NHC(═S)CH₂Hal, and —(C═O)C₁₋₃alkyl;
R^{b}, independently, is selected from the group consisting of hydrogen, C₁₋₆alkyl, aryl, heteroaryl, C₃₋₈cycloalkyl, and heterocycloalkyl;
R^{c}, independently, is selected from the group consisting of hydrogen, (C═O)CH₃, C₁₋₆alkyl, CF₃, CH₂F, and aryl, or two R^{c} groups are taken together with the carbon to which they attached to form a C₃₋₈cycloalkyl group; and
R^{e} is NH₂ or OH;
or a pharmaceutically acceptable salt, hydrate, or prodrug thereof.

2. The compound of group 1 wherein Y is null,

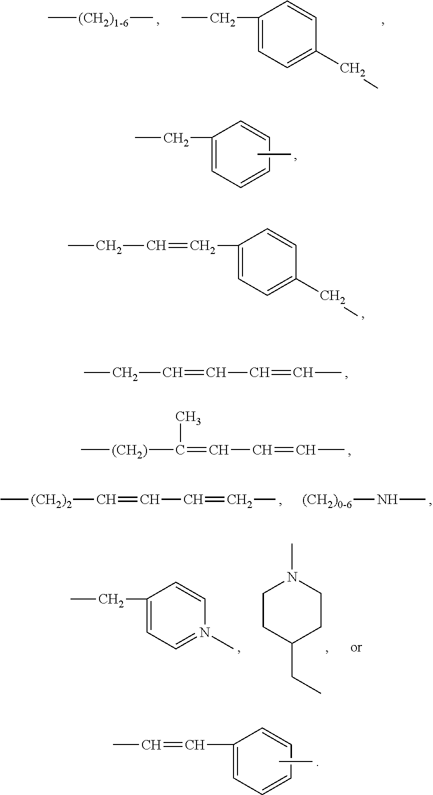

3. The compound of group 1 wherein Z is

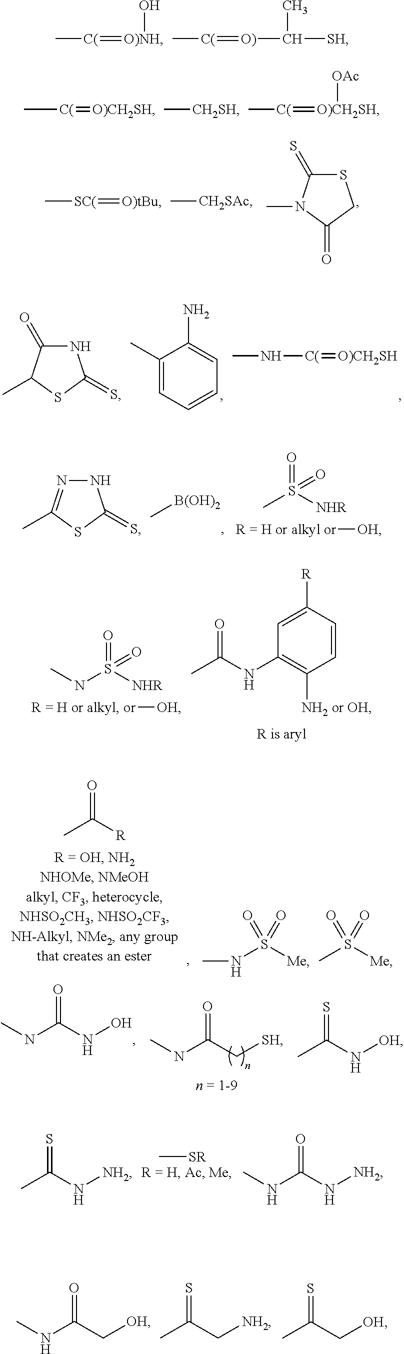

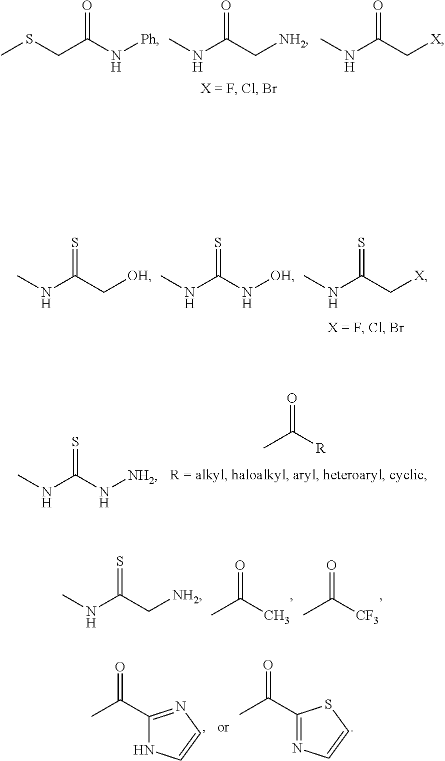

4. A compound selected from the group consisting of 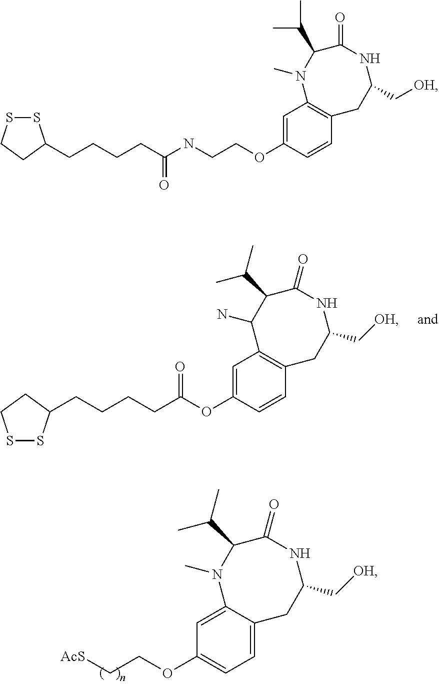
wherein n is an integer 2 through 9.

5. A pharmaceutical composition comprising (a) compound of claim 1, (b) an optional second therapeutic agent useful in the treatment of Alzheimer's Disease, and (c) an excipient and/or pharmaceutically acceptable carrier.

6. A method of treating a disease or condition wherein activation of PKC and inhibition of HDAC provides a benefit comprising administering a therapeutically effective amount of a compound of claim 1 to an individual in need thereof.

7. The method of claim 6 further comprising administering a therapeutically effective amount of a second therapeutic agent useful in the treatment of the disease or condition.

8. The method of claim 7 wherein the compound of claim 1 and the second therapeutic agent are administered simultaneously.

9. The method of claim 7 wherein the compound of claim 1 and the second therapeutic agent are administered separately.

10. The method of claim 6 wherein the disease or condition is a neurological disease.

11. The method claim 10 wherein the neurological disease is selected from the group consisting of Huntington's disease, lupus, schizophrenia, multiple sclerosis, muscular dystrophy, dentatorubralpallidoluysian atrophy, spinal and bulbar muscular atrophy, fine spinocerebellar ataxias, drug-induced movement disorders, Creutzfeldt-Jakob disease, amyotrophic lateral sclerosis, Pick's disease, Alzheimer's disease, Lewy body dementia, cortico basal degeneration, dystonia, myoclonus, Tourette's syndrome, tremor, chorea, restless leg syndrome, Parkinson's disease, Parkinsonian syndromes, anxiety, depression, psychosis, manic depression, Friedreich's ataxia, Fragile X syndrome, spinal muscular dystrophy, Rett syndrome, Rubinstein-Taybi syndrome, Wilson's disease, and multi-infarct state.

12. The method of claim 10 wherein the neurological disease or condition is Alzheimer's Disease.

13. The method of claim 7, wherein the disease or condition is Alzheimer's Disease, and the second therapeutic agent is memantine, galantamine, rivastigmine, donepezil, tacrine, or a mixture thereof.
